(19) United States
(12) Patent Application Publication
(10) Pub. No.: US 2023/0241001 A1
(43) Pub. Date: Aug. 3, 2023

(54) COMPOSITIONS AND METHODS COMPRISING IONIZABLE LIPID NANOPARTICLES ENCAPSULATING BARCODED MRNA

(71) Applicants: The Trustees of the University of Pennsylvania, Philadelphia, PA (US); Cornell University, Ithaca, NY (US)

(72) Inventors: Michael Mitchell, Philadelphia, PA (US); Pedro Guimaraes, Belo Horizonte (BR); Rui Zhang, Philadelphia, PA (US); Roman Spektor, Cambridge, MA (US)

(21) Appl. No.: 17/761,274

(22) PCT Filed: Sep. 19, 2020
(86) PCT No.: PCT/US2020/051684

§ 371 Date: Mar. 17, 2022

Related U.S. Application Data
(60) Provisional application No. 62/903,391, filed on Sep. 20, 2019.

(51) Int. Cl. A61K 9/51; A61K 31/7105; C12N 15/11; G01N 33/574

(52) U.S. Cl. CPC A61K 9/5123; A61K 31/7105; C12N 15/11; G01N 33/57492; A61K 9/5146

(57) ABSTRACT

Provided herein are compositions comprising a lipid nanoparticle (LNP) formulation comprising a LNP having encapsulated therein a barcoded mRNA (b-mRNA), and methods for using the same. Such methods include analyzing in vivo delivery of a composition.

### CROSS-REFERENCE TO RELATED APPLICATIONS

This is a National Stage Entry under 35 U.S.C. 371 of International Patent Application No. PCT/US2020/051684, filed Sep. 19, 2020, which claims benefit of provisional application No. 62/903,391, filed Sep. 20, 2019. These applications are incorporated by reference herein.

### STATEMENT OF GOVERNMENT INTEREST

This invention was made with government support under TR002776 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “UPN-19-9058PCT_Seq-Listing_ST25.txt”, having a size of 39.9 kB and dated Sep. 22, 2020.

### BACKGROUND

Messenger RNA (mRNA), which offers amplified production of therapeutic proteins through rapid and repeated translation in cells, has recently garnered significant attention as a therapeutic for the treatment or prevention of a range of diseased⁻⁷. This is due, in part, to significant improvements in in vitro transcription that have enabled the development of mRNA with high potency, low-cost manufacturing, and low innate immunogenicity for in vivo delivery⁸⁻⁹. mRNA offers several advantages over the delivery of DNA to produce therapeutic proteins. One major benefit is that mRNA does not need to cross the nuclear barrier in cells to induce protein expression⁹. Therefore, mRNA can be transfected more efficiently than plasmid DNA, especially for slowly dividing cells¹⁰. Additionally, by not needing to reach to the cell nucleus, mRNA does not bear the risk of insertional mutagenesis and carcinogenesis associated with genomic integration. mRNA delivery also offers several advantages over the direct delivery of proteins, as the large size, instability, and high production costs of proteins hinder their use in vivo¹¹. The promise of mRNA as a new class of genetic medicine has led to significant investments in its commercial development—including companies such as Moderna, CureVac AG, and BioNTech¹¹⁻¹³—with ongoing clinical trials focused on vaccination, cancer immunotherapy, and protein replacement¹⁴⁻¹⁶. While significant progress has been made in the design of in vitro-transcribed mRNA, the widespread use of mRNA as a therapeutic requires safe and effective delivery technologies⁶. mRNA is 10⁵-10⁶ Dalton in size and approximately three to four orders of magnitude larger than small molecules that diffuse into cells⁶. Furthermore, mRNA is highly negatively charged and thus repulses the anionic cell membrane⁸. Naked mRNA is also inherently unstable and quickly degraded by RNases⁸.

Ionizable lipid nanoparticles (LNPs) have been engineered to encapsulate and protect nucleic acids—including mRNA—from degradation and mediate their intracellular delivery¹⁷. Of note, an LNP-based small interfering RNA (siRNA) drug developed by Alnylam was approved by the U.S. Food and Drug Administration in 2018¹⁸. Several potent ionizable lipids have been synthesized using various approaches, including rational design approaches where the lipid head and tail structures are systematically varied¹⁹⁻²³, as well as through the creation of large combinatorial libraries of lipid-like materials²⁴⁻²⁶. In addition to an ionizable lipid, LNPs are commonly formulated with three excipients: (i) cholesterol, which enhances the stability of the LNP bilayer and promotes membrane fusion²⁷; (ii) a phospholipid, which fortifies the LNP bilayer structure and also aids in endosomal escape²⁸; and (iii) a lipid-polyethylene glycol (PEG) conjugate, which inserts into the LNP bilayer and provides a PEG coating that reduces LNP aggregation and nonspecific endocytosis by immune cells²⁹.

While LNPs have demonstrated significant promise for nucleic acid delivery applications, their therapeutic potential is limited by inefficient delivery to target cells and tissues in vivo. This is due, in part, to an incomplete understanding of how LNP physicochemical properties affect in vivo delivery³⁰. The effects of LNP physicochemical properties are typically characterized and screened in an in vitro or ex vivo setting, and LNP structural and pKa criteria have been shown to predict delivery to particular organs in vivo^{17,21}. However, it is challenging to fully replicate the biological barriers that affect the biological fate of LNPs in vivo—including anatomical structures, circulating cells, and physiological forces—in in vitro and ex vivo experiments³⁰.

Approaches that facilitate the screening of LNPs in an in vivo setting may enhance our fundamental understanding of how LNP structure affects in vivo mRNA delivery to target cells and tissues. Recently, new approaches have emerged to facilitate the screening of nanoparticles (NPs) in an in vivo setting, leveraging various technologies including mass cytometry, DNA barcoding (b-DNA), and high-throughput sequencing³¹⁻³³. In the context of mass cytometry—where current instruments permit up to 50 metal isotope labels to be detected simultaneously in single cells—approaches have been developed to enable high-throughput quantification of gold nanoparticles in single cells as a means to identify novel nanoparticle-based vaccines to target dendritic cells in vivo³¹. In addition to mass cytometry, b-DNA in tandem with PCR and deep sequencing has been utilized to accelerate drug discovery³⁴. Rather than testing compounds individually, many DNA tagged compounds can be administered in a single pool, and compounds that interact with the target can be identified by their b-DNA using deep sequencing³⁴. This b-DNA concept has recently been applied to LNP delivery, in the context of identifying barcoded NPs that target tumors³², as well as those that deliver nucleic acid therapeutics in vivo^{33,35}.

What is needed in the art are technologies that facilitate the screening of LNPs in an in vivo setting can enhance our fundamental understanding of how LNP structure affects in vivo mRNA delivery to target cells and tissues.

### SUMMARY OF THE INVENTION

In the context of mRNA LNP delivery screening in vivo, an ideal approach may leverage a functional mRNA with a barcoded region in its 3′ untranslated region (UTR) that can be quantified using deep sequencing, rather than the encapsulation of additional b-DNA that may potentially alter LNP structure and subsequent in vivo delivery³⁵⁻³⁶. Towards this end, we have designed an in vivo delivery platform consisting of a library of engineered LNPs that encapsulate functional, custom-designed barcoded mRNA (b-mRNA, FIG. 1). These b-mRNA are similar in structure and function to regular mRNA, and contain barcodes and unique molecular identifier (UMI) that enables LNP in vivo delivery to be quantified via deep sequencing (FIG. 1B). We formulated a mini-library of LNPs via microfluidic mixing, where each LNP formulation encapsulated a unique b-mRNA (FIG. 1C). We show that different b-mRNA LNP formulations can be pooled together, simultaneously administered intravenously into mice, and LNP delivery to multiple organs can be quantified using deep sequencing (FIG. 1D). Subsequently, deep sequencing results were validated via LNP delivery of commercially available luciferase mRNA, indicating that this platform can be utilized to effectively identify lead LNP formulations for mRNA delivery in vivo to organs such as the liver and spleen. Additionally, we show that different types of nucleic acid cargo (b-mRNA versus a DNA barcode) altered LNP delivery in vivo, suggesting that the incorporation of different nucleic acid barcode structures within LNPs can affect their in vivo fate. This delivery platform, where functional mRNAs are barcoded and encapsulated in LNPs, can accelerate in vivo screening and the design of LNPs for mRNA gene therapy. Provided herein, in one aspect is a composition comprising a lipid nanoparticle (LNP) formulation. The formulation includes a LNP having encapsulated therein a barcoded mRNA (b-mRNA). In one embodiment, the b-mRNA includes one or more of a PCR handle at the 3′ UTR, a barcode sequence, and a unique molecular identifier (UMI).

In another aspect, a method of analyzing in vivo delivery of a composition is provided. The method includes providing at least one LNP formulation to a subject and identifying the barcode sequence of the b-mRNA in one or more tissues of the subject, thereby correlating the specific LNP formulation found in the tissue by the identification of the barcode. In one embodiment, the method further includes detecting the presence of the reporter molecule in one or more tissues of the subject, quantifying the b-mRNA in the one or more tissues, and/or sorting cells from the multiple tissues of the subject based on the presence or absence of the reporter molecule, wherein the cells having the reporter molecule are also sorted based on the presence or absence of a cell surface protein that is indicative of tissue type or cell type.

In yet another aspect, a method of determining a personalized treatment for a subject is provided. The method includes obtaining a biological sample from the subject, said sample containing one or more cells; providing at least one LNP formulation to the sample; and identifying the barcode sequence of the b-mRNA in the one or more cells, thereby correlating the specific LNP formulation found in the tissue by the identification of the barcode.

Other aspects and advantages of the invention will be readily apparent from the following detailed description of the invention.

### BRIEF DESCRIPTION OF THE DRAWINGS

FIGS. 1A-1E provide a schematic of lipid nanoparticles (LNPs) encapsulating barcoded mRNA (b-mRNA) for accelerated in vivo delivery screening. (A) b-mRNA was generated via in vitro transcription (IVT) with a plasmid vector template coding for the luciferase reporter gene luc2. (B) b-mRNA includes a region coding for luciferase, a barcode sequence, a 10-nucleotide unique molecular identifier (UMI), and a poly(A) tail. (C) LNPs were formulated via microfluidic mixing, and each LNP formulation encapsulated unique b-mRNA. (D) Different LNP formulations were then pooled together and administered intravenously to C57BL/6 mice. Organs were harvested 4 hours post injection, and b-mRNA delivery was quantified using both whole-organ luminescence imaging and deep sequencing.

FIGS. 2A-2C demonstrate formulation and characterization of b-mRNA LNPs. (A) LNPs were formulated via microfluidic mixing of an aqueous phase of b-mRNA and an ethanol phase of ionizable lipids, phospholipids, cholesterol, and a lipid-polyethylene glycol (PEG) conjugate. (B) Representative cryogenic-transmission electron microscopy image of LNPs encapsulating b-mRNA (scale bar=100 nm). (C) Hydrodynamic diameter measurements of LNPs encapsulating b-mRNA quantified by dynamic light scattering.

FIGS. 3A-3E show that b-mRNA LNPs accelerate in vivo liver and spleen delivery screening and the identification of lead formulations. (A) LNP formulations with identical lipid and excipient composition but different b-mRNA were pooled at varying dosages and administered intravenously to C57BL/6 mice. 4 hours post injection, delivery of each b-mRNA LNP to the liver was quantified. N=4 mice per group. (B) In vivo standard curve of b-mRNA delivery to the liver at a range of dosages showed a linear regression (R2=310.9646). (C-E) LNP formulations (F01-F16) were engineered by varying the content of ionizable lipid, phospholipid (DOPE), cholesterol, and lipid-anchored PEG (C14-PEG2000). A 0.25 μg dose of each b-mRNA LNP was pooled and administered intravenously as a single injection. 4 hours post injection, b-mRNA delivery to the liver (C), spleen (D), and other organs (E) were quantified. N=4 mice per group. Heat map (E) representing delivery to different tissue samples were created using Morpheus software. Darker clusters were designated as higher delivery whereas lighter clusters were designated as lower delivery. Data plotted as mean±SD. Method to calculate b-mRNA delivery is explained in detail in the experimental section. R2 value was calculated based on linear regression model.

FIGS. 4A-4C show the lead LNPs identified from the delivery screen induce greater in vivo luciferase expression in liver and spleen. C57BL/6 mice were intravenously injected with either LNP formulations F01 or F13 (5 μg b-mRNA per injection). 4 hours post administration, organs were harvested from mice, and their luminescence was measured by IVIS imaging. N=3 mice per group. (A) Representative images of luminescence detection in organs from mice treated with either LNP formulations F01 or F13. (B, C) Total luminescent flux from two organs of interest, the liver and spleen, were quantified in (B) and (C) respectively. Data were plotted as mean±SD. N.S. denotes not significant, **P<0.01 by t-test.

FIGS. 5A-5C show LNPs encapsulating widely used, commercially available luciferase mRNA are comparable in vivo to b-mRNA LNPs. 5 different LNP formulations (F01, F06, F09, 13 F13, F16) were formulated with commercially available luciferase mRNA (Trilink-mRNA). C57BL/6 mice were intravenously injected with individual LNP formulations (5 μg Trilink mRNA per injection). 4 hours post administration, organs were harvested from mice, and their luminescence was measured by IVIS imaging. N=3 mice per group. (A) Representative images of luminescence detection in organs from mice treated with 5 different LNP formulations (F01, F06, F09, F13, F16). (B, C) Total luminescent flux from two organs of interest, the liver and spleen, were quantified in (B) and (C) respectively. Data were plotted as mean±SD. N.S. denotes not significant, *P<0.05, ***P<0.001, ****P<0.0001 by ANOVA with post-hoc Tukey-Kramer.

FIGS. 6A-6E show encapsulation of barcoded DNA (b-DNA) versus b-mRNA in LNPs alters in vivo delivery. (A) 16 LNP formulations used in this study were now used to each encapsulate unique b-DNA instead of b-mRNA. b-DNA contained universal primer sites, a 10-nucleotide barcode sequence, and a 10-nucleotide UMI region to minimize polymerase chain reaction (PCR) bias. (B-C) 16 b-DNA LNP formulations were pooled (1 μg b-DNA per injection for each formulation) and administered to C57BL/6 mice intravenously. 4 hours post injection, b-DNA delivery to the liver (B) and spleen (C) was quantified. N=4 mice per group. (D-E) In vivo delivery of 16 b-mRNA LNP formulations was plotted against the delivery of 16 b-DNA LNP formulations. Method to calculate b-DNA delivery is explained in detail in the experimental section. R2 values were calculated based on a linear regression model. Data were plotted as mean±SD.

FIGS. 7A-7D show a comparison of b-mRNA system versus b-DNA system to predict functional mRNA delivery in vivo (A, B) b-mRNA LNP delivery was plotted against luciferase expression in the liver (A) and spleen (B) of luciferase mRNA LNP-treated mice. (C, D) Similarly, b-DNA LNP delivery was plotted against luciferase expression in the liver (C) and spleen (D) of luciferase mRNA LNP-treated mice. Data were plotted as mean±SD.

FIGS. 8A-8C demonstrate barcoded mRNA (b-mRNA) optimization and quality control. (A) bEnd.3 cells were treated with LNPs encapsulating different b-mRNA (modified with either pseudouridine (w) or 5-methylcytosine (m5C)) or commercially available Trilink-mRNA at different concentration. Luciferase activity was assessed at 48 hours after treatment. (BSC) Representative Bioanalyzer trace of b-mRNA from different batches.

FIGS. 9A and 9B. Encapsulation of barcoded DNA (b-DNA) versus b-mRNA in LNPs alters LNP physical properties. Hydrodynamic diameter and PDI of LNPs encapsulating b-mRNA or b-DNA were analyzed by dynamic light scattering and were shown in (A) and (B) respectively.

FIGS. 10A-10F. F2, F14, F15, and F16 are more efficient than other LNP formulations to deliver barcoded mRNA (b-mRNA) to the brain (10A), lung (10B), heart (10C), kidney (10D), pancreas (10E), and muscle (10F). 16 b-mRNA LNP formulations were pooled and injected into C57BL/6 mice intravenously (0.25 μg of each b-mRNA was included in a single injection). 4 hours post injection, b-mRNA delivery to the lung, kidney, heart, pancreas, brain, and muscle were quantified. N=4 mice per group. Data were plotted as mean±SD.

### DETAILED DESCRIPTION OF THE INVENTION

Messenger RNA (mRNA) has recently emerged as a promising class of nucleic acid therapy, with the potential to induce protein production to treat and prevent a range of diseases. While significant progress has been made in the design of in vitro-transcribed mRNA with high potency, low-cost manufacturing, and low innate immunogenicity, the widespread use of mRNA as a therapeutic requires safe and effective in vivo delivery technologies. Libraries of ionizable LNPs have been designed to encapsulate mRNA, prevent its degradation and mediate intracellular delivery. However, these LNPs are typically characterized and screened in an in vitro setting, which may not fully replicate the biological barriers that they encounter in vivo.

Here, an in vivo platform to accelerate mRNA delivery screening is provided, consisting of a library of engineered LNPs that encapsulate functional, custom-designed barcoded mRNA (b-mRNA). These b-mRNA are similar in structure and function to regular mRNA, and contain barcodes that enable their delivery to be quantified via deep sequencing. Using mini-library of b-mRNA LNPs formulated via microfluidic mixing, these different formulations can be pooled together, administered intravenously into mice as a single pool, and their delivery to multiple organs (liver, spleen, brain, lung, heart, kidney, pancreas, and muscle) can be quantified simultaneously using deep sequencing. In the context of liver and spleen delivery, LNPs that exhibited high b-mRNA delivery also yielded high luciferase expression, indicating that this platform can identify lead LNP candidates as well as optimal formulation parameters for in vivo mRNA delivery. Interestingly, LNPs with identical formulation parameters that encapsulated different types of nucleic acid barcodes (b-mRNA versus a DNA barcode) altered in vivo delivery, suggesting that the structure of the barcoded nucleic acid affects LNP in vivo delivery. This platform, which enables direct barcoding and subsequent quantification of a functional mRNA itself, can accelerate the in vivo screening and design of LNPs for mRNA therapeutic applications such as CRISPR/Cas9 gene editing, mRNA vaccination, and other mRNA-based regenerative medicine and protein replacement therapies.

Provided herein are compositions which include a lipid nanoparticle (LNP) formulation comprising a LNP having encapsulated therein a barcoded mRNA (b-mRNA), and methods for utilizing same.

Barcoded mRNA (b-mRNA)

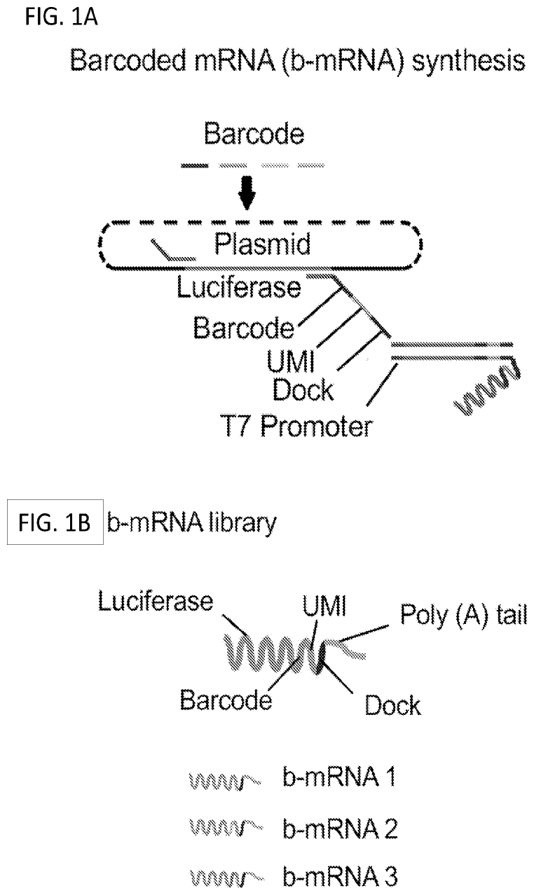

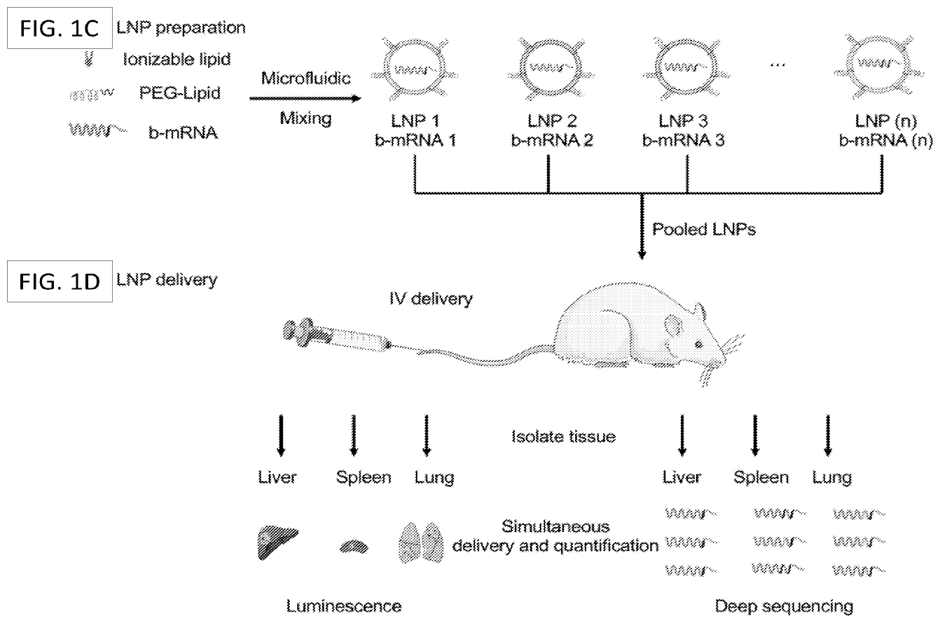

The LNP formulations described herein incorporate barcoded mRNAs (b-mRNAs) which allow for identification and quantification of in vivo delivered mRNAs. As used herein, messenger RNA (mRNA) refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. The barcoded mRNA or b-mRNA includes, in some embodiments, one or more of (i) a PCR handle at the 3′ UTR, (ii) a unique “barcode” sequence; and (iii) a unique molecular identifier (UMI), which are further described below. See FIGS. 1A and 1B.

In one embodiment, the b-mRNA includes a “handle” or “dock” sequence. The handle is a nucleic acid sequence which serves as a dock for downstream polymerase chain reaction (PCR) amplification. The handle is a unique sequence that is not present in the host genome.

The b-mRNA includes a barcode sequence. This sequence is a unique sequence which allows identification of the specific b-mRNA being tested or employed. The barcode sequence also allows for quantification of the in vitro transcribed b-mRNA during analysis by deep sequencing. The barcode can be designed to any length available using synthesis technology, and the length of the barcode limits the number of formulations that may be tested simultaneously. For example, using the 10 bp barcode exemplified herein, there are a total of 1048576 possible combinations. Thus, the barcode sequence is, in one embodiment, between 5 nt to 100 nt in length. In another embodiment, the barcode sequence is between 10 nt to 20 nt in length. In one embodiment, the barcode is 10 nt in length. In another embodiment, the barcode is 5, 6, 7, 8, 9, 10, 11, 12, 13, 14, 15, 16, 17, 18, 19 or 20 nt in length.

In one embodiment, the b-mRNA includes a unique molecular identifier (UMI) to identify each individual b-mRNA. The UMI are randomly generated sequences which serve to avoid duplication during deep sequencing. Inclusion of these UMI in the first steps of sequencing library preparation offers several benefits. UMI create a distinct identity for each input molecule; this makes it possible to estimate the efficiency with which input molecules are sampled, identify sampling bias, and most importantly, identify and correct for the effects of PCR amplification bias. The UMI can be designed to any length available using synthesis technology. The UMI is, in one embodiment, between 5 nt to 100 nt in length. In another embodiment, the UMI is between 10 nt to 20 nt in length. In one embodiment, the UMI is 10 nt in length. In another embodiment, the UMI is 5, 6, 7, 8, 9, 10, 11, 12, 13, 14, 15, 16, 17, 18, 19 or 20 nt in length. Design of UMI is known in the art, for example, Clement et al, AmpUMI: design and analysis of unique molecular identifiers for deep amplicon sequencing, Bioinformatics, Volume 34, Issue 13, 1 Jul. 2018, Pages i202-i210 which is incorporated herein by reference.

The b-mRNA molecule also includes a coding sequence for a protein of interest. The protein of interest may also be a reporter molecule, which is capable of being identified and/or measured. In one embodiment, as exemplified herein, the reporter molecule is luciferase. Luciferase (Luc) is commonly used in mammalian cell culture to measure both gene expression and cell viability. It emits bioluminescence in the presence of the substrate, luciferin. In another embodiment, the reporter molecule is Green Fluorescent Protein. GFP is a commonly used direct detection reporter in mammalian cell culture, yielding bright green fluorescence with an emission peak at 509 nm. In another embodiment, the reporter is mCherry. mCherry is derived from DsRed, a protein found in Discosoma sp. mCherry is a monomeric fluorophore with a peak absorption at 587 nm and emission at 610 nm. It is photostable and resistant to photobleaching. In another embodiment, the reporter is β-galactosidase (β-gal), encoded by the LacZ gene. β-gal catalyzes the conversion of β-galactosides into monosaccharides. It is a common marker gene used to assess transfection efficiency. Other reporter genes are known in the art and are useful herein.

The protein of interest may be a biologically active molecule, such as a therapeutic protein. As used herein, the term “biologically active” refers to a characteristic of any agent that has activity in a biological system, and particularly in an organism. For instance, an agent that, when administered to an organism, has a biological effect on that organism, is considered to be biologically active. In certain embodiments, provided herein are compositions comprising mRNA that encode one or more prophylactically- or therapeutically-active proteins, polypeptides, or other factors. For example, the mRNA may encode an agent that enhances tumor killing activity (such as TRAIL or tumor necrosis factor (TNF)) in a cancer. As additional non-limiting example, the mRNA may encode an agent suitable for the treatment of conditions such as muscular dystrophy (a suitable mRNAs encodes Dystrophin), cardiovascular disease (suitable mRNAs encode, e.g., SERCA2a, GATA4, Tbx5, Mef2C, Hand2, Myocd, etc.), neurodegenerative disease (suitable mRNAs encode, e.g., NGF, BDNF, GDNF, NT-3, etc.), chronic pain (suitable mRNAs encode GlyRa1, an enkephalin, or a glutamate decarboxylase (e.g., GAD65, GAD67, or another isoform), lung disease (e.g., CFTR), hemophilia (suitable mRNAs encode, e.g., Factor VIII or Factor IX), neoplasia (suitable mRNAs encode, e.g., PTEN; ATM; ATR; EGFR; ERBB2; ERBB3; ERBB4; Notch1; Notch2; Notch3; Notch4; AKT; AKT2; AKT3; HIF; H1Fla; HIF3a; Met; HRG; Bc12; PPARalpha; PPAR gamma; WT1 (Wilms Tumor); FGF Receptor Family members (5 members: 1, 2, 3, 4, 5); CDKN2a; APC; RB (retinoblastoma); MEN!; VHL; BRCA1; BRCA2; AR (Androgen Receptor); TSG101; IGF; IGF Receptor; IgfI (4 variants); Igf2 (3 variants); IgfI Receptor; Igf2 Receptor; Bax; Bc12; caspases family (9 members: 1, 2, 3, 4, 6, 7, 8, 9, 12); Kras; Apc), age-related macular degeneration (suitable mRNAs encode, e.g., Aber; Cc12; Cc2; cp (ceruloplasmin); Timp3; cathepsinD; Vldlr), schizophrenia (suitable mRNAs encode, e.g. Neuregulinl (NrgI); Erb4 (receptor for Neuregulin); ComplexinI (CplxI); TphI Tryptophan hydroxylase; Tph2 Tryptophan hydroxylase 2; Neurexin 1; GSK3; GSK3a; GSK3b; 5-HIT (Slc6a4); COMT; DRD (DrdIa); SLC6A3; DAOA; DTNBPI; Dao (Dao1)), trinucleotide repeat disorders (suitable mRNAs encode, e.g., HTT (Huntington's Dx); SBMA/SMAXI/AR (Kennedy's Dx); FXN/X25 (Friedrich's Ataxia); ATX3 (Machado-Joseph's Dx); ATXNI and ATXN2 (spinocerebellar ataxias); DMPK (myotonic dystrophy); Atrophin-1 and Atn1(DRPLA Dx); CBP (Creb-BP-global instability); VLDLR (Alzheimer's); Atxn7; Atxn10), fragile X syndrome (suitable mRNAs encode, e.g., FMR2; FXRI; FXR2; mGLUR5), secretase related disorders (suitable mRNAs encode, e.g., APH-1 (alpha and beta); Presenilin (Psen1); nicastrin (Ncstn); PEN-2), ALS (suitable mRNAs encode, e.g., SOD1; ALS2; STEX; FUS; TARD BP; VEGF (VEGF-a; VEGF-b; VEGF-c)), autism (suitable mRNAs encode, e.g. Mecp2; BZRAP1; MDGA2; SemaSA; Neurexin 1; Fragile X (suitable mRNAs encode, e.g., FMR2; AFF2; FXR1; FXR2; Mglur5), Alzheimer's disease (suitable mRNAs encode, e.g., E1; CHIP; UCH; UBB; Tau; LRP; PICALM; Clusterin; PS1; SORL1; CR1; Vldlr; Uba1; Uba3; CHIP28 (Aqp1, Aquaporin 1); Uchl1; Uch13; APP), inflammation (suitable mRNAs encode, e.g., IL-10; IL-1 (IL-Ia; IL-Ib); IL-13; IL-17 (IL-17a (CTLA8); IL-17b; IL-17c; IL-17d; IL-171); 11-23; Cx3cr1; ptpn22; TNFa; NOD2/CARD15 for IBD; IL-6; IL-12 (IL-12a; IL-12b); CTLA4; Cx3c11, Parkinson's Disease (suitable mRNAs encode, e.g., x-Synucicin; DJ-1; LRRK2; Parkin; PINK1), blood and coagulation disorders, such as, e.g., anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, and thalassemia (suitable mRNAs encode, e.g., CRAN1, CDA1, RPS19, DBA, PKLR, PK1, NT5C3, UMPH1, PSNI, RHAG, RH50A, NRAMP2, SPTB, ALAS2, ANH1, ASB, ABCB7, ABC7, ASAT, TAPBP, TPSN, TAP2, ABCB3, PSF2, RING11, MHC2TA, C2TA, RFX5, RFXAP, RFX5, TBXA2R, P2RX1, P2X1, HF1, CFH, HUS, MCFD2, FANCA, FACA, FA1, FA, FAA, FAAP95, FAAP90, F1134064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1), B-cell non-Hodgkin lymphoma or leukemia (suitable mRNAs encode, e.g., BCL7A, BCL7, ALI, TCLS, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AML1, WHSC1LI, NSD3, FLT3, AF1Q, NPM1, NUMA1, ZNF145, PLZF, PML, MYL, STAT5B, AF1Q, CALM, CLTH, ARL11, ARLTS1, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN), inflammation and immune related diseases and disorders (suitable mRNAs encode, e.g., KIR3DL1, NKAT3, NKB1, AMB11, K1R3DS1, IFNG, CXCL12, TNFRSF6, APT1, FAS, CD95, ALPS1A, IL2RG, SCIDX1, SCIDX, IMD4, CCL5, SCYA5, D17S136E, TCP228, IL10, CSIF, CMKBR2, CCR2, CMKBR5, CCCKR5 (CCR5), CD3E, CD3G, AICDA, AID, HIGM2, TNFRSF5, CD40, UNG, DGU, HIGM4, TNFSFS, CD40LG, HIGM1, IGM, FOXP3, IPEX, AIID, XPID, PIDX, TNFRSF14B, TACI), inflammation (suitable mRNAs encode, e.g., IL-10, IL-1 (IL-Ia, IL-Ib), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cII); JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, IMD4), metabolic, liver, kidney and protein diseases and disorders (suitable mRNAs encode, e.g., TTR, PALB, APOA1, APP, AAA, CVAP, AD1, GSN, FGA, LYZ, TTR, PALB, KRT18, KRT8, CIRH1A, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, SCOD1, SCO1, CTNNB1, PDGFRL, PDGRL, PRLTS, AX1NI, AXIN, CTNNB1, TP53, P53, LFS1, IGF2R, MPRI, MET, CASP8, MCH5, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, SEC63), muscular/skeletal diseases and disorders (suitable mRNAs encode, e.g., DMD, BMD, MYF6, LMNA, LMN1, EMD2, FPLD, CMDIA, HGPS, LGMDIB, LMNA, LMNI, EMD2, FPLD, CMD1A, FSHMD1A, FSHD1A, FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD1C, SEPN1, SELN, RSMD1, PLEC1, PLTN, EBS1, LRP5, BMND1, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1), neurological and neuronal diseases and disorders (suitable mRNAs encode, e.g., SOD1, ALS2, STEX, FUS, TARDBP, VEGF (VEGF-a, VEGF-b, VEGF-c), APP, AAA, CVAP, AD1, APOE, AD2, PSEN2, AD4, STM2, APBB2, FE65LI, NOS3, PLAU, URK, ACE, DCPI, ACEI, MPO, PAC1PI, PAXIPIL, PTIP, A2M, BLMH, BMH, PSEN1, AD3, Mecp2, BZRAP1, MDGA2, Sema5A, Neurexin 1, GLO1, MECP2, RTT, PPMX, MRX16, MRX79, NLGN3, NLGN4, KIAA1260, AUTSX2, FMR2, FXR1, FXR2, mGLUR5, HD, IT15, PRNP, PRIP, JPH3, JP3, HDL2, TBP, SCA17, NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, NDUFV2, MECP2, RTT, PPMX, MRX16, MRX79, CDKL5, STK9, MECP2, RTT, PPMX, MRX16, MRX79, x-Synuclein, DJ-1, Neuregulinl (NrgI), Erb4 (receptor for Neuregulin), ComplexinI (Cplx1), TphI Tryptophan hydroxylase, Tph2, Tryptophan hydroxylase 2, Neurexin 1, GSK3, GSK3a, GSK3b, 5-HTT (Slc6a4), CONT, DRD (Drd1a), SLC6Ar3, DADA, DTNBP1, Dao (Dao1), APH-1(alpha and beta), Presenilin (Psen1), nicastrin, (Ncstn), PEN-2, Nos1, Parp1, Nat1, Nat2, HTT, SBMA/SMAX1/AR, FXN/X25, ATX3, TXN, ATXN2, DMPK, Atrophin-1, Atn1, CBP, VLDLR, Atxn7, and Atxn10), and ocular diseases and disorders (suitable mRNAs encode, e.g., Aber, Cc12, Cc2, cp (ceruloplasmin), Timp3, cathepsinD, Vldlr, Ccr2, CRYAA, CRYA1, CRYBB2, CRYB2, PITX3, BFSP2, CP49, CP47, CRYAA, CRYAI, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, KRITL APOA1, TGFBI, CSD2, CDGG1, CSD, BIGH3, CDG2, TACSTD2, TROP2, MIST, VSX1, RINX, PPCD, PPD, KTCN, COL8A2, FECD, PPCD2, PIP5K3, CFD, KERA, CNA2, MYOC, TIGR, GLCIA, JOAG, GPOA, OPTN, GLC1E, FIP2, HYPL, NRP, CYP1BI, GLC3A, OPA1, NTG, NPG, CYP1BI, GLC3A, CRB1, RP12, CRX, CORD2, CRD, RPGRIPI, LCA6, CORD9, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, and VMD2).

In certain embodiments, the b-mRNA encodes a factor that can affect the differentiation of a cell. For example, expression of one or more of Oct4, Klf4, Sox2, c-Myc, L-Myc, dominant-negative p53, Nanog, Glis1, Lin28, TFIID, mir-302/367, or other miRNAs can cause the cell to become an induced pluripotent stem (iPS) cell. See also, Takahashi and Yamanaka, Cell, 126: 663-676 (2006); Takahashi, Cell, 131: 861-872 (2007); Wernig, Nature, 448: 318-324 (2007); and Yu, Science, 318: 1917-1920 (2007), the disclosures of which are incorporated herein by reference. Alternatively, the mRNA may encode a factor for transdifferentiating cells (e.g., one or more of GATA4, Tbx5, Mef2C, Myocd, Hand2, SRF, Mesp1, SMARCD3 (for cardiomyocytes); Ascl1, Nurr1, Lmx1A, Brn2, Mytl1, NeuroD1, FoxA2 (for neural cells), Hnf4a, Foxa1, Foxa2 or Foxa3 (for hepatic cells).

The b-mRNA may be generated using in vitro transcription (IVT). DNA templates are generated, and IVT is performed, using techniques known in the art. For example, for the experiments described herein, DNA templates were designed that included the following components: (i) a T7 promoter in the 5′ untranslated region (UTR) to initiate in vitro transcription, (ii) a PCR handle at the 3′ UTR for downstream polymerase chain reaction (PCR) amplification, (iii) a barcode sequence for quantification of in vitro transcribed b-mRNA during analysis by deep sequencing, and (iv) a unique molecular identifier (UMI) to avoid duplication during deep sequencing (FIG. 1A). The sequences of the IVT templates used herein are shown in Table 4.

The DNA templates were used for in vitro transcription to produce b-mRNA with dual functions: (i) the luciferase sequence enables b-mRNA to be translated and produce luciferase protein, (ii) the barcode and UMI sequences enable identification and quantification of b-mRNA through deep sequencing (FIG. 1B). Due to the ease of output measurements, luciferase mRNA has become one of the most commonly utilized sequences for gene delivery. Therefore, in one embodiment, luciferase mRNA is utilized as the target sequence for the b-mRNA design.

It is known that mRNA modifications can enhance stability while suppressing innate immune responses and subsequently improving transfection⁴²⁻⁴⁵. Therefore, provided herein are b-mRNA with various modifications. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. A typical mRNA molecule has a 5′ end and a 3′ end. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In one embodiment, the b-mRNA incorporates pseudouridine (w). In another embodiment, the b-mRNA incorporates 5-methylcytosine (m5C).

Lipid Nanoparticle Formulation

The clinical and scientific potential of nucleic acid therapies is limited by inefficient drug delivery to target cells. Drug delivery vehicles must avoid clearance by the immune and reticuloendothelial systems, access the correct organ, and enter specific cells within a complex tissue microenvironment. At each of these steps, anatomical structures and biological molecules can actively engage the vehicles and influence their final destination. It is not currently possible to recapitulate the totality of this complex process in cell culture. As described herein, LNPs are utilized with b-mRNAs in a novel system to effectively evaluate in vivo mRNA delivery.

In one aspect, provided herein are LNP formulations. LNPs useful herein are known in the art. As used herein, LNPs are comprised of cholesterol (aids in stability and promotes membrane fusion), a phospholipid (which provides structure to the LNP bilayer and also may aid in endosomal escape), a polyethylene glycol (PEG) derivative (which reduces LNP aggregation and “shields” the LNP from non-specific endocytosis by immune cells), and an ionizable lipid (complexes negatively charged RNA and enhances endosomal escape), which form the LNP-forming composition. Fenton et al, Bioinspired Alkenyl Amino Alcohol Ionizable Lipid Materials for Highly Potent in vivo mRNA Delivery, Adv Mater. 2016 Apr. 20; 28(15): 2939-2943, which is incorporated herein by reference.

The various components of the LNP-forming composition may be selected based on the desired target, cargo, size, etc. For example, previous studies have shown that that polymeric nanoparticles made of low molecular weight polyamines and lipids can deliver nucleic acids to endothelial cells with high efficiency. Dahlman, et al, In vivo endothelial siRNA delivery using polymeric nanoparticles with low molecular weight, Nat Nanotechnol. 2014 August; 9(8): 648-655, which is incorporated herein by reference in its entirety.

The LNP-forming composition includes an ionizable lipid or lipid-like material. As exemplified herein, in one embodiment, the ionizable lipid is C12-200. In another embodiment, the ionizable lipid is CKK-E12. In another embodiment, the ionizable lipid is 5A2-SC8. In another embodiment, the ionizable lipid is BAMEA-O16B. In another embodiment, the ionizable lipid is 306O₁₀. In another embodiment, the ionizable lipid is 7C1. See, Love et al, Lipid-like materials for low-dose, in vivo gene silencing, Proceedings of the National Academy of Sciences February 2010, 107 (5) 1864-1869; Dong et al, Lipopeptides and selective siRNA delivery, Proceedings of the National Academy of Sciences March 2014, 111 (11) 3955-3960; Cheng et al, Dendrimer-Based Lipid Nanoparticles Deliver Therapeutic FAH mRNA to Normalize Liver Function and Extend Survival in a Mouse Model of Hepatorenal Tyrosinemia Type I, Advanced Materials, 30(52) (December 2018); Liu et al, Fast and Efficient CRISPR/Cas9 Genome Editing In Vivo Enabled by Bioreducible Lipid and Messenger RNA Nanoparticles, Advanced Materials, 31(33), August 2019; and Hajj et al, Branched-Tail Lipid Nanoparticles Potently Deliver mRNA In Vivo due to Enhanced Ionization at Endosomal pH, Small, 15(6) (February 2019), each of which are incorporated herein by reference. Other ionizable lipids are known in the art and are useful herein.

The LNP-forming composition includes phospholipid. As exemplified herein, in one embodiment, the phospholipid (helper) is DOPE. In another embodiment, the phospholipid is DSPC. In another embodiment, the phospholipid is DOTAP. In another embodiment, the phospholipid is DOTMA. See, Kauffman et al, Optimization of Lipid Nanoparticle Formulations for mRNA Delivery in Vivo with Fractional Factorial and Definitive Screening Designs, Nano Lett., October 2015, 15(11):7300-7306; Blakney et al, Inside out: optimization of lipid nanoparticle formulations for exterior complexation and in vivo delivery of saRNA, Gene Therapy, July 2019; and Patel et al, Lipid nanoparticles for delivery of messenger RNA to the back of the eye, J Controlled Release, 303:91-100, (June 2019), each of which are incorporated herein by reference. Other phospholipids are known in the art and are useful herein. As described by Kauffman et al, cited above, incorporation of DOPE is desirable for LNP formulations carrying mRNA.

The LNP-forming composition includes a PEG derivative. As exemplified herein, in one embodiment, the PEG derivative is a lipid-anchored PEG. In one embodiment, the lipid-anchored PEG is C14-PEG2000. In another embodiment, the lipid-anchored PEG is C14-PEG1000. In another embodiment, the lipid-anchored PEG is C14-PEG3000. In another embodiment, the lipid-anchored PEG is C14-PEG5000. In another embodiment, the lipid-anchored PEG is C12-PEG1000. In another embodiment, the lipid-anchored PEG is C12-PEG2000. In another embodiment, the lipid-anchored PEG is C12-PEG3000. In another embodiment, the lipid-anchored PEG is C12-PEG5000. In another embodiment, the lipid-anchored PEG is C16-PEG1000. In another embodiment, the lipid-anchored PEG is C16-PEG2000. In another embodiment, the lipid-anchored PEG is C16-PEG3000. In another embodiment, the lipid-anchored PEG is C16-PEG5000. In another embodiment, the lipid-anchored PEG is C18-PEG1000. In another embodiment, the lipid-anchored PEG is C18-PEG2000. In another embodiment, the lipid-anchored PEG is C18-PEG3000. In another embodiment, the lipid-anchored PEG is C18-PEG5000.

The LNP formulations comprising the LNPs having encapsulated therein a b-mRNA are formed using techniques known in the art. For example, an organic solution containing the lipids is mixed together with an acidic aqueous solution containing the b-mRNA in a microfluidic channel resulting in the formation of mRNA-loaded LNPs.

The compositions provided herein may include multiple LNP formulations as described above. In one embodiment, each LNP formulation includes a b-mRNA having a uniquely identifiable nucleotide barcode sequence. The unique barcode provides the ability to identify the specific LNP which produces the desired result. The LNP formulation may also differ in the LNP-forming composition used to generate the LNP. For example, the LNP-forming compositions can be varied in the molar amount and/or structure of the ionizable lipid, the molar amount and/or structure of the helper lipid, the molar amount/or structure of PEG, and/or the molar amount of cholesterol. Additionally, or alternatively, the LNP formulation may comprise b-mRNAs which differ in the coding sequence for the biologically active molecule. Additionally, or alternatively, the LNP formulation may comprise b-mRNAs which differ in the modifications made to the mRNA.

In some embodiments, a composition as described herein, is administered to a subject. Administration can be through a number of routes. In one embodiment, administration is intravenous. In another embodiment, administration is oral. Routes of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration may be intrathecal, intraventricular, intraocular (subretinal or intravitreal), intracerebroventricular (ICV), intra-cisterna magna (ICM) or intracranial.

In some embodiments, the composition comprises a pharmaceutically acceptable carrier. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In another aspect, also provided are methods of using the compositions described herein. As described in detail herein, the compositions are useful to accelerate mRNA delivery screening. Various b-mRNA LNPs are formulated via microfluidic mixing, pooled together, and administered intravenously into a subject as a single pool. Delivery to multiple organs (liver, spleen, brain, lung, heart, kidney, pancreas, and muscle) can be quantified simultaneously using deep sequencing.

In one embodiment, a method of analyzing in vivo delivery of a composition is provided. The method includes providing at least one LNP formulation as described herein to a subject and identifying the barcode sequence of the b-mRNA in one or more tissues of the subject. The specific LNP formulation found in the tissue is determined based on the identification of the barcode. The barcode can be identified using known PCR amplification techniques. For example, the barcode can be identified using deep sequencing as described, e.g., by Dahlman, et al, In vivo endothelial siRNA delivery using polymeric nanoparticles with low molecular weight, Nat Nanotechnol. 2014 August; 9(8): 648-655, which is incorporated herein by reference in its entirety. In addition, these techniques are useful to quantify the amount of b-mRNA in one or more tissues of the subject.

In another embodiment, the method includes detecting the presence of the reporter molecule in one or more tissues of the subject. The reporter molecule can be detected using known methods appropriate for the reporter gene chosen, e.g., luciferase expression readouts. The reporter molecule also allows for functional assessment of the expression level of the b-mRNA. In the context of screening, this serves as an indicator of the expression level of the specific LNP formulation in vivo, as it may be translated to a biologically active molecule. The cells from the multiple tissues of the subject may be sorted based on the presence or absence of the reporter molecule.

In yet another embodiment, the cells having the reporter molecule are also sorted based on the presence or absence of a cell surface protein that is indicative of tissue type or cell type. These individual cells or groups of cells may be further analyzed to determine the particular b-mRNA present therein.

It is intended that, in some embodiments, more than one LNP formulation is administered to the subject. Using techniques known in the art, a large number of LNP formulations can be assessed in a single experiment. The examples herein describe the use of up to 16 LNP formulations at once. However, this number is limited by the size of the barcode (as discussed above) and a greater number of LNP formulations are contemplated herein. In one embodiment, about 5 to about 50,000 LNP formulations are assayed at once. In another embodiment, about 50 to about 5000 LNP formulations are assayed at once. In one embodiment, about 50 to about 500 LNP formulations are assayed at once. In one embodiment, about 5 LNP formulations are assayed at once. In another embodiment, about 50 LNP formulations are assayed at once. In another embodiment, about 500 LNP formulations are assayed at once. In another embodiment, about 5000 LNP formulations are assayed at once. In another embodiment, about 50,000 LNP formulations are assayed at once. The methods described herein are particularly suited for high-throughput assays.

As noted above, each LNP formulation includes a b-mRNA having a uniquely identifiable nucleotide barcode sequence. The LNP formulation may also differ in the LNP-forming composition used to generate the LNP. Additionally, or alternatively, the LNP formulation may comprise b-mRNAs which differ in the coding sequence for the biologically active molecule. Additionally, or alternatively, the LNP formulation may comprise b-mRNAs which differ in the modifications made to the mRNA.

In yet another aspect, a method of determining a personalized treatment for a subject is provided. The method includes obtaining a biological sample from the subject, the sample containing one or more cells; providing at least one LNP formulation to the sample; and identifying the barcode sequence of the b-mRNA in the one or more cells. The specific LNP formulation found in the tissue is determined by the identification of the barcode. The sample may be any biological sample that contains cells of interest. For example, the sample may be a tissue sample. In another embodiment, the sample is a tumor biopsy. In yet another embodiment, the sample is a blood sample.

As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In another embodiment, the subject is a mouse.

The dosage of the LNP composition to be delivered to a patient can be determined by the person of skill in the art. Such dosages range from 0.1 μg or less to 1000 μg including endpoints and all numbers therebetween. In one embodiment, the dosage ranges from 0.1 to 10 μg. In another embodiment, the dosage ranges from 0.4-10 μg. In another embodiment, the dosage ranges from 0.5-100 μg. In yet another embodiment, the dosage ranges from 50 to 500 μg. All ranges include endpoints and all numbers therebetween.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1: Discussion

Barcoded mRNA (b-mRNA) Synthesis

b-mRNA LNP Formulation and Characterization: To synthesize in vitro transcribed b-mRNA (FIG. 1A), DNA templates were designed that included the following four necessary components: (i) a T7 promoter in the 5′ untranslated region (UTR) to initiate in vitro transcription, (ii) a PCR handle at the 3′ UTR for downstream polymerase chain reaction (PCR) amplification, (iii) a barcode sequence for quantification of in vitro transcribed b-mRNA during analysis by deep sequencing, and (iv) a unique molecular identifier (UMI) to avoid duplication during deep sequencing (FIG. 1A).

The DNA templates were used for in vitro transcription to produce b-mRNA with dual functions: (i) the luciferase sequence enables b-mRNA to be translated and produce luciferase protein, (ii) the barcode and UMI sequences enable identification and quantification of b-mRNA through deep sequencing (FIG. 1B). Due to the ease of output measurements, luciferase mRNA has become one of the most commonly utilized sequences for gene delivery. Therefore, in one embodiment, luciferase mRNA is utilized as the target sequence for the b-mRNA design.

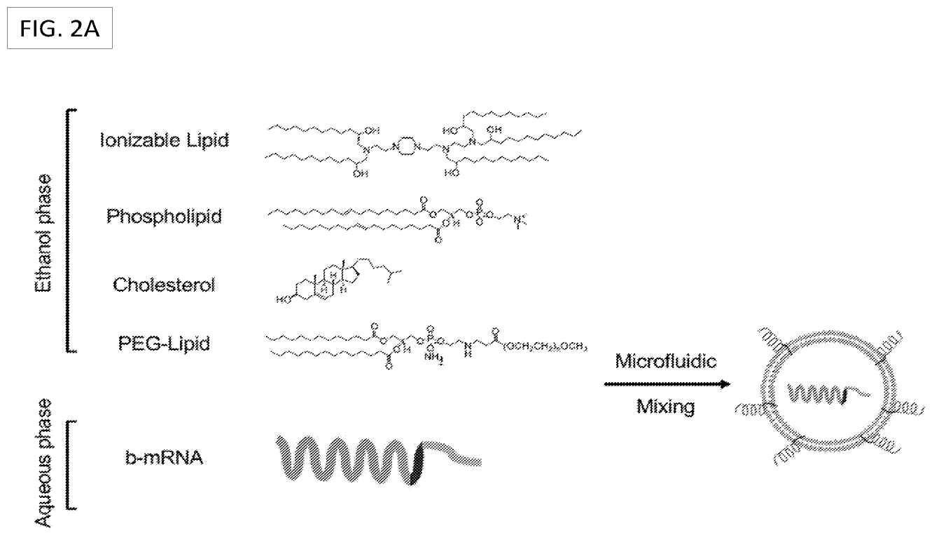

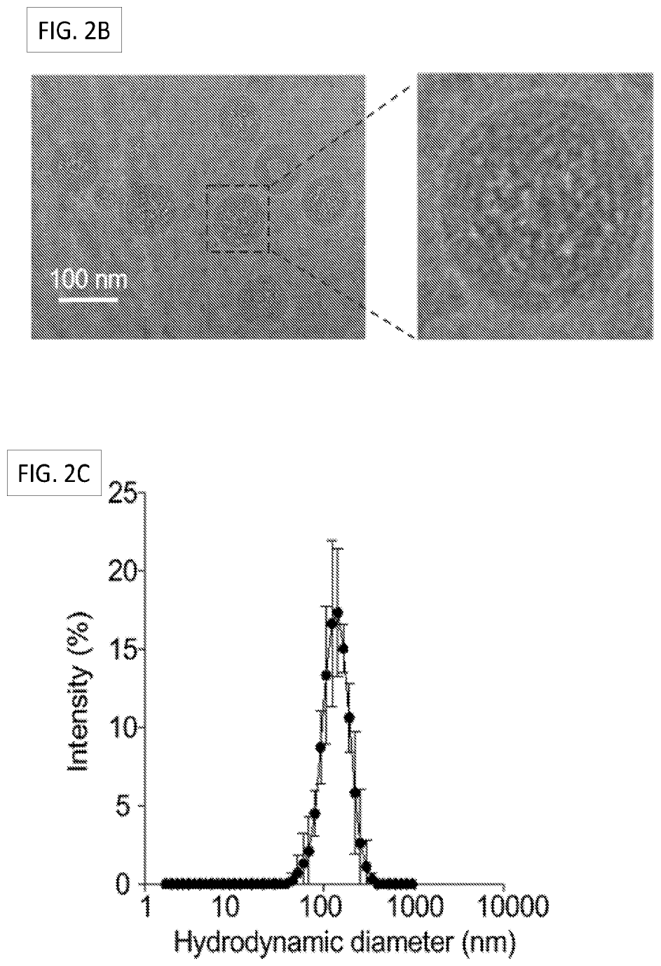

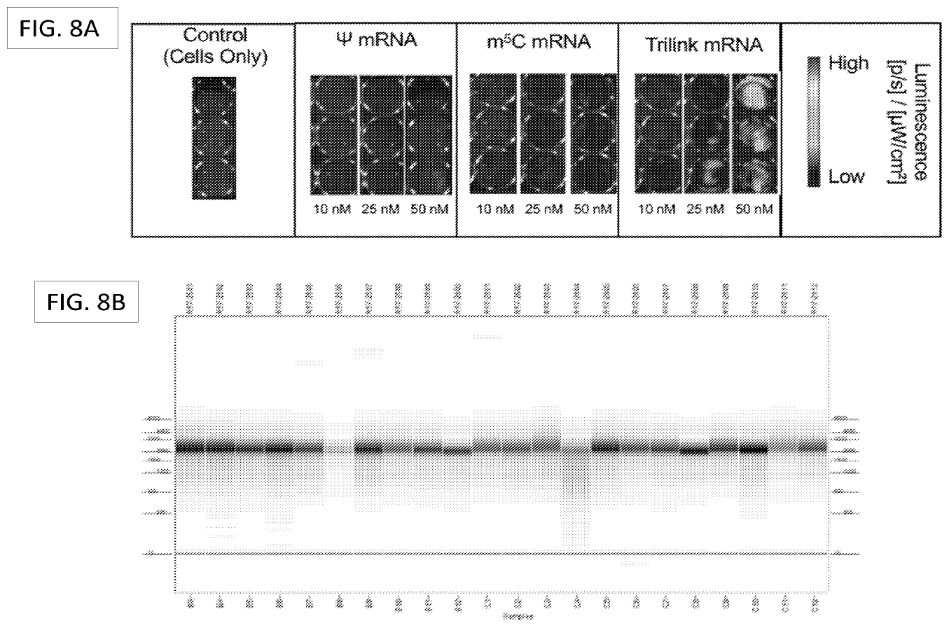

Previous research demonstrates that mRNA modifications can enhance stability while suppressing innate immune responses and subsequently improving transfection⁴²⁻⁴⁵. Therefore, to assess the potency of b-mRNA with various modifications, we performed in vitro transcription to produce b-mRNA containing two different modifications, pseudouridine (w) or 5-methylcytosine (m5C). To compare the transfection efficiency between w modified b-mRNA and m5C modified b-mRNA, both b-mRNA were encapsulated in a previously optimized LNP for mRNA delivery⁴⁶. Additionally, a commercially available mRNA (Trilink-mRNA) was encapsulated in an identical formulation and included as a positive control. In vitro transfection results showed that m5C modified b-mRNA induced higher luciferase expression than w modified b-mRNA (FIG. 8A). Therefore, we performed all subsequent experiments using m5C modified b-mRNA, unless otherwise noted. The b-mRNA synthesis protocol was shown to be reproducible, and in vitro transcribed m5C b-mRNA consistently produced full-length polyadenylated transcripts with minimum batch-to-batch variability (FIG. 8B and FIG. 2, FIG. 8C).

In Vivo LNP Delivery and Dose-Dependent b-mRNA Detection

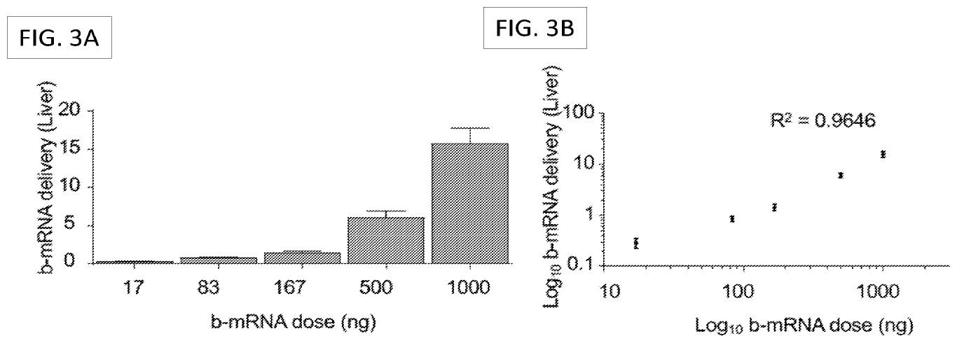

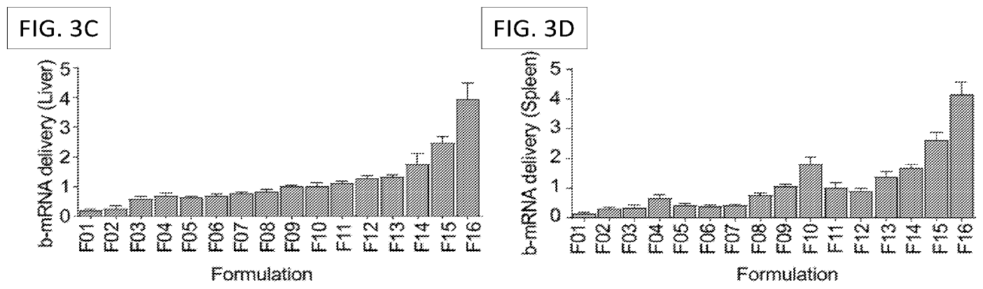

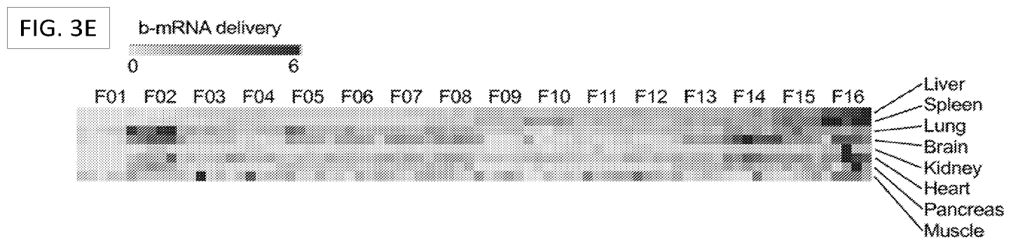

To assess whether b-mRNA can be delivered in vivo to the liver of mice and quantified using deep sequencing, five identical LNP formulations that each encapsulated different b-mRNA were pooled together—at different mRNA doses for each LNP formulation (17-1000 ng mRNA per LNP formulation)—and administered intravenously via tail vein injection into mice. 4 hours post-injection, the liver was harvested from mice and LNP b-mRNA delivery was quantified using deep sequencing. LNP formulations that delivered doses as low as 17 ng of total b-mRNA were detected using deep sequencing (FIG. 3A), indicating that b-mRNA delivered using LNPs can be quantified at low doses. In a recent study, LNPs were systemically injected into mice at total mRNA doses as high as −80 μg (4 mg/kg)¹⁹. Given that we were able to detect LNP doses as low as 17 ng total b-mRNA via deep sequencing, this platform can potentially allow for several thousand unique b-mRNA LNP formulations to be administered into mice and screened for delivery. b-mRNA LNP delivery was also shown to be dose-dependent, as LNPs delivered at a lower total b-mRNA dose resulted in lower sequencing reads and overall delivery to the liver, while LNPs delivered at higher doses resulted in higher sequencing reads and overall delivery (FIG. 3A). Furthermore, a linear correlation (R²=0.9646) between barcode delivery to the liver and total b-mRNA dose administered (FIG. 3B) was found, further indicating that the b-mRNA quantification using deep sequencing is accurate. These results indicate that (i) unique b-mRNA LNPs can be pooled together, delivered in vivo, and quantified using deep sequencing, and (ii) deep sequencing can be used to accurately quantify b-mRNA LNP delivery across a broad range of injected b-mRNA doses.

Simultaneous Delivery Screening of Multiple b-mRNA LNP Formulations In Vivo

After demonstrating the feasibility of the delivery system, we investigated whether several different b-mRNA LNP formulations can be screened simultaneously for in vivo delivery to various organs in mice. We formulated a mini-library of 16 different LNPs that were previously evaluated for in vivo mRNA delivery⁴⁶—now encapsulating b-mRNA instead of mRNA—as a means to validate the b-mRNA LNP screening platform (Table 1).

TABLE 1
Formulation details for all LNPs

| | Ionizable | Molar ratio % | | | |
|---|---|---|---|---|---|
| | lipid:mRNA | Ionizable | Phospho- | | PEG- |
| Formulation | ratio | lipid | lipid | Cholesterol | Lipid |
| F01 | 2.5:1 | 40 | 4 | 53.5 | 2.5 |
| F02 | 2.5:1 | 60 | 10 | 29.5 | 0.5 |
| F03 | 7.5:1 | 40 | 28 | 28.5 | 3.5 |
| F04 | 10:1 | 40 | 28 | 29.5 | 2.5 |
| F05 | 12.5:1 | 40 | 22 | 35.0 | 3.0 |
| F06 | 7,5:1 | 35 | 22 | 40.0 | 3.0 |
| F07 | 10:1 | 35 | 22 | 39.5 | 3.5 |
| F08 | 10:1 | 30 | 16 | 51.0 | 3.0 |
| F09 | 5:1 | 30 | 16 | 51.5 | 2.5 |
| F10 | 7.5:1 | 40 | 16 | 42.5 | 1.5 |
| F11 | 5:1 | 35 | 16 | 46.5 | 2.5 |
| F12 | 7.5:1 | 35 | 16 | 46.5 | 2.5 |
| F13 | 10:1 | 35 | 16 | 46.5 | 2.5 |
| F14 | 12.5:1 | 35 | 16 | 46.5 | 2.5 |
| F15 | 15:1 | 35 | 16 | 46.5 | 2.5 |
| F16 | 25:1 | 35 | 16 | 46.5 | 2.5 |

16 different b-mRNA LNP formulations were characterized by hydrodynamic diameter, polydispersity, and encapsulation efficiency (Table 2). The hydrodynamic diameter of all LNPs were between 74.42 nm and 90.77 nm, while their polydispersity ranged from 0.174 to 0.233 (Table 2). 13 of the 16 formulations possessed surface charge values between 0 mV and −10 mV, while the remaining 3 formulations had greater negative charge values (between −10 mV and −20 mV) (Table 2). Additionally, efficient b-mRNA encapsulation rates (over 85%) were observed in 11 of the 16 LNP formulations (Table 2).

TABLE 2
Characterization of b-mRNA encapsulated LNPs

| | Hydrodynamic | | Zeta Potential | Encapsulation |
|---|---|---|---|---|
| Formulation | Diameter (nm) | PDI | (mV) | efficiency (%) |
| F01 | 82.40 | 0.198 | −14.70 | 67.5 |
| F02 | 90.77 | 0.226 | −16.70 | 33.7 |
| F03 | 78.53 | 0.224 | −2.65 | 48.9 |
| F04 | 81.49 | 0.202 | −5.76 | 92.8 |
| F05 | 74.42 | 0.226 | −2.10 | 95.7 |
| F06 | 82.03 | 0.221 | −3.39 | 90.7 |
| F07 | 79.23 | 0.212 | −4.34 | 49.9 |
| F08 | 83.25 | 0.205 | −5.08 | 91.3 |
| F09 | 81.40 | 0.174 | −9.04 | 92.7 |
| F10 | 75.58 | 0.208 | −8.16 | 89.1 |
| F11 | 83.55 | 0.203 | −11.70 | 89.8 |
| F12 | 88.09 | 0.224 | −7.53 | 88.4 |
| F13 | 83.36 | 0.204 | −3.77 | 87.4 |
| F14 | 77.32 | 0.214 | −4.23 | 76.6 |
| F15 | 84.71 | 0.233 | −4.22 | 88.7 |
| F16 | 80.85 | 0.223 | −4.01 | 86.1 |

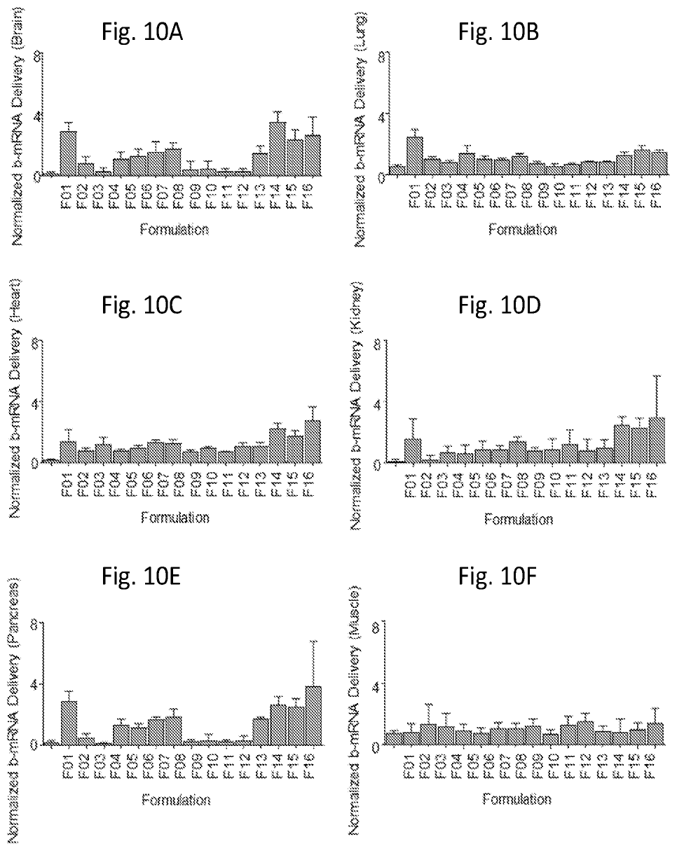

The 16 LNP formulations, each containing a unique b-mRNA, were then pooled and injected intravenously into mice at a dose of 0.25 μg total b-mRNA for each LNP formulation. 4 hours post-injection, organs (the liver, spleen, lung, brain, kidney, heart, pancreas, and muscle) were harvested from mice and LNP b-mRNA delivery was quantified using deep sequencing. We found a broad range in delivery of different b-mRNA LNPs to the liver (FIG. 3C), spleen (FIG. 3D), and other tissues (FIG. 3E and FIG. 10).

Some LNPs demonstrated similar behavior regarding b-mRNA delivery to different tissues. For example, F14-F16 showed higher b-mRNA delivery to most tissues (the liver, spleen, brain, heart, kidney, and pancreas) compared to other LNPs. Additionally, F11 to F16 were formulated with C12-200:b-mRNA weight ratios varying between 5:1 to 25:1, and we observed enhanced b-mRNA delivery to the liver and spleen with increased C12-200:mRNA ratios. Together, these data confirm that the b-mRNA platform can be used to screen several different LNP formulations in vivo simultaneously, and potentially identify lead LNPs for optimal mRNA delivery. While this mini-library of 16 LNP formulations serves as a proof-of-concept to validate the in vivo screening approach, the ease of synthesizing unique b-mRNA can be exploited to formulate and screen larger libraries of b-mRNA LNPs in vivo.

b-mRNA LNP Delivery Measurements are Comparable to LNP-Mediated mRNA Transfection In Vivo

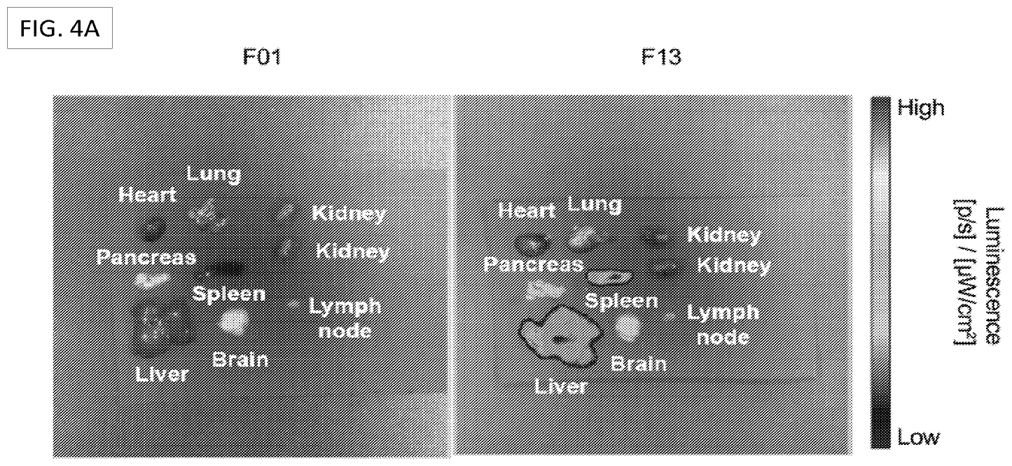

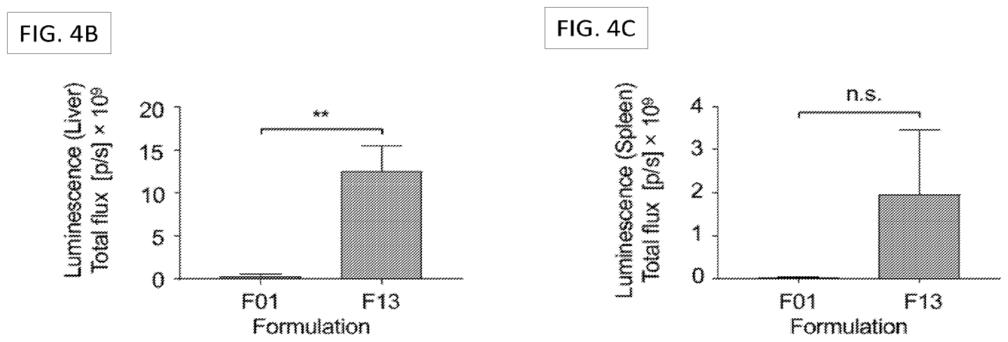

Given that b-mRNA was also designed to encode for the reporter protein firefly luciferase, we next assessed whether our relative b-mRNA LNP delivery measurements from deep sequencing were similar to functional in vivo luciferase expression readouts in mice. Two LNP formulations (F01 and F13) were selected for in vivo luciferase expression studies (FIG. 4), as F13 had higher relative b-mRNA delivery to the liver and spleen than F01 (FIG. 3C). Therefore, if the b-mRNA in vivo screen is accurate in terms of relative LNP delivery measurements, mice injected with F13 should have higher luciferase expression in the liver and spleen than those injected with F01 at the same total b-mRNA dose. F01 and F13 were separately administrated to two groups of mice, and 4 hours post-injection luciferase expression from different organs was quantified by an in vivo imaging system (IVIS, FIG. 4A). Similar to a previous study⁴⁶, high luciferase expression was observed in the liver and spleen (FIG. 4A). Luciferase expression in the liver (FIG. 4B) and spleen (FIG. 4C) was higher in mice treated with F13—which had higher relative b-mRNA delivery to the liver and spleen—than mice treated with F01. Collectively, these results indicate that (i) b-mRNA LNPs can be quantified for relative delivery using deep sequencing and functional luciferase expression using IVIS, (ii) LNPs that yield higher relative delivery—measured using deep sequencing—also induced higher luciferase expression, and (iii) this screening platform can potentially be utilized to predict b-mRNA LNP transfection in vivo.

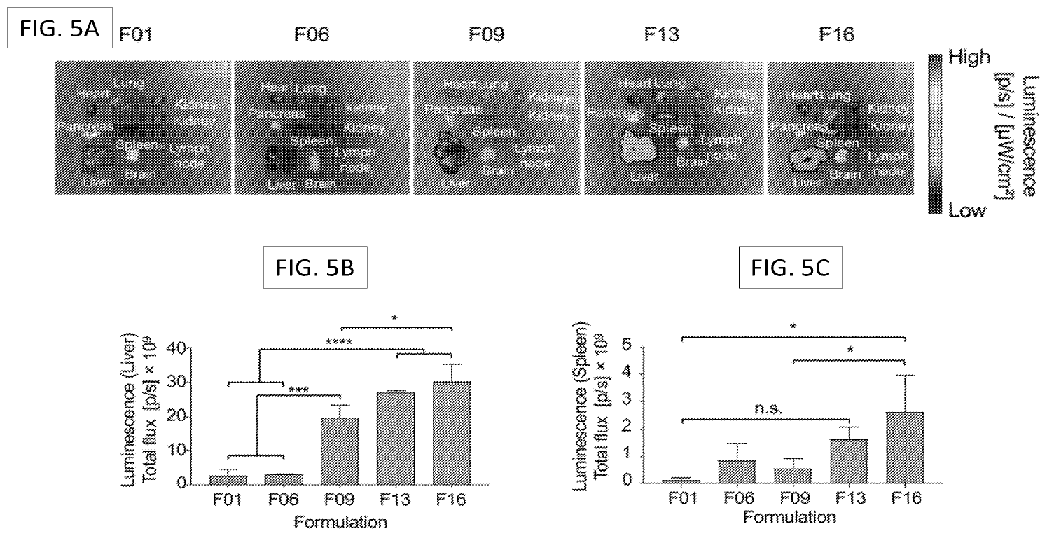

To further validate our screening platform for functional mRNA delivery, LNPs from the initial screen were formulated with a commercially available mRNA encoding for luciferase (Trilink-mRNA)21, 38-40. Two LNPs with low relative b-mRNA delivery (F01, F06) and three LNPs with high relative delivery (F09, F13, F16) were then formulated with Trilink-mRNA encoding for firefly luciferase and were injected separately into 5 groups of mice. 4 hours post-injection, 8 tissue samples (liver, spleen, kidney, lung, brain, pancreas, heart, and muscle) were harvested, and luciferase expression was measured (FIG. 5A). Similar to previous in vivo luciferase studies with b-mRNA LNPs (FIG. 4), F13 encapsulating Trilink-mRNA induced higher luciferase expression than F01 encapsulating Trilink-mRNA in both the liver (FIG. 5B) and spleen (not statistically significant, P=0.149; FIG. 5C). These results suggest that LNPs encapsulating commercially available mRNA are comparable to b-mRNA LNPs in terms of function, and can potentially be utilized to validate results from in vivo b-mRNA LNP screening.

Barcoded Nucleic Acid Structure Impacts LNP Biodistribution In Vivo

The structure of different nucleic acid cargo (e.g. DNA, siRNA, mRNA) encapsulated within LNPs has previously been shown to play an important role in the LNP formulation process, requiring different types and ratios of ionizable lipids and excipients that consequently affect the physical properties of LNPs^{21, 48-50}. A recent study showed that when different nucleic acid cargo (i.e. siRNA or mRNA) were encapsulated in the same LNP formulation, dramatic changes were found in terms of LNP size as well as the spatial location of various components (e.g. cholesterol, helper lipid, and PEG)′, indicating that the structure of the nucleic acid cargo encapsulated within LNPs ultimately affects LNP structure. However, how these structural changes can affect LNP delivery in vivo was not studied in the same report. Therefore, we compared our b-mRNA system to a previously studied b-DNA system to assess how different therapeutic cargos 1 (i.e. b-DNA versus b-mRNA) affect LNP delivery⁵¹.

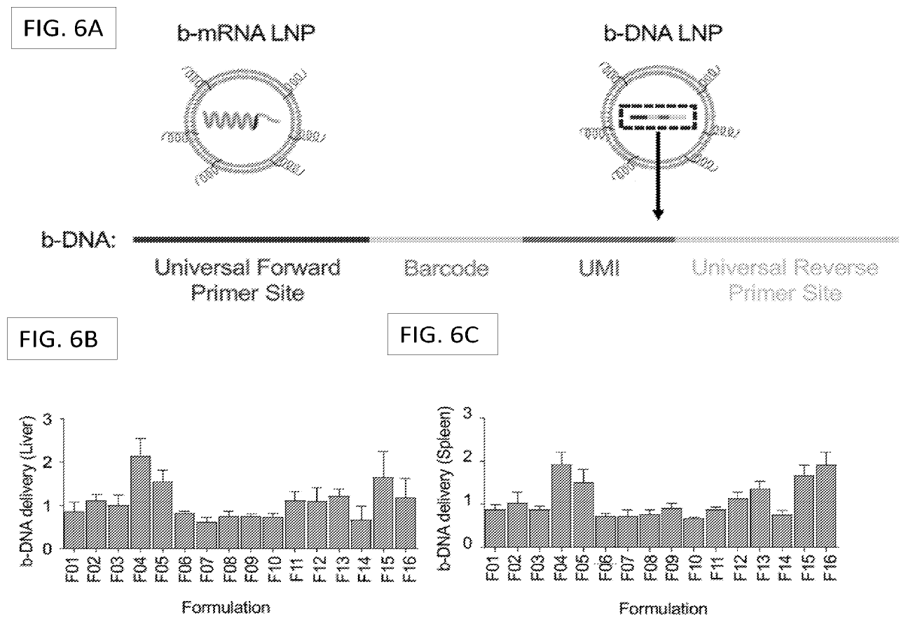

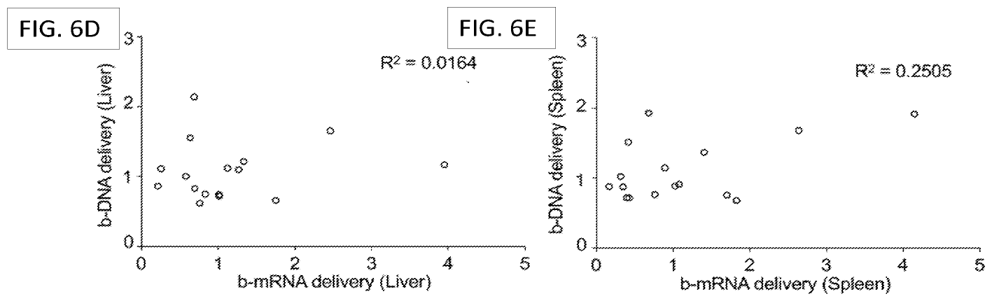

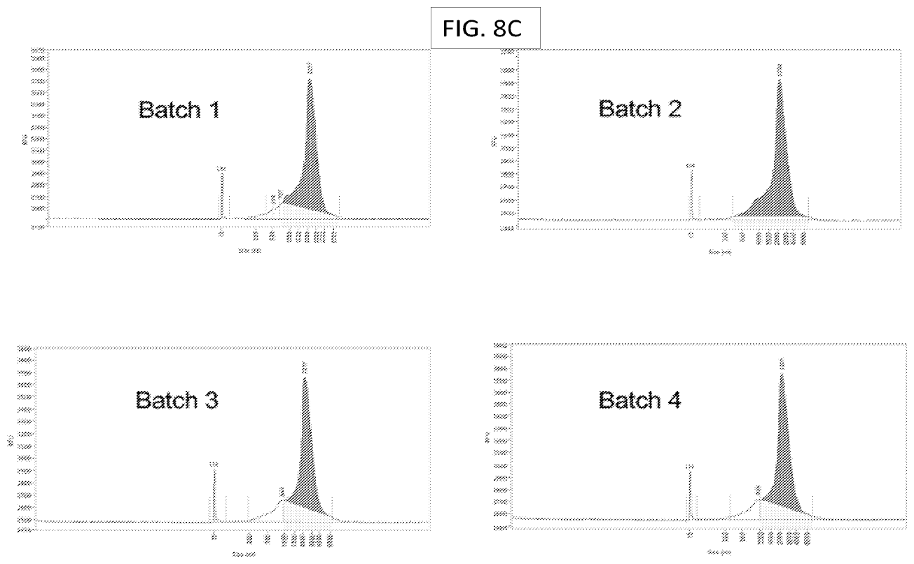

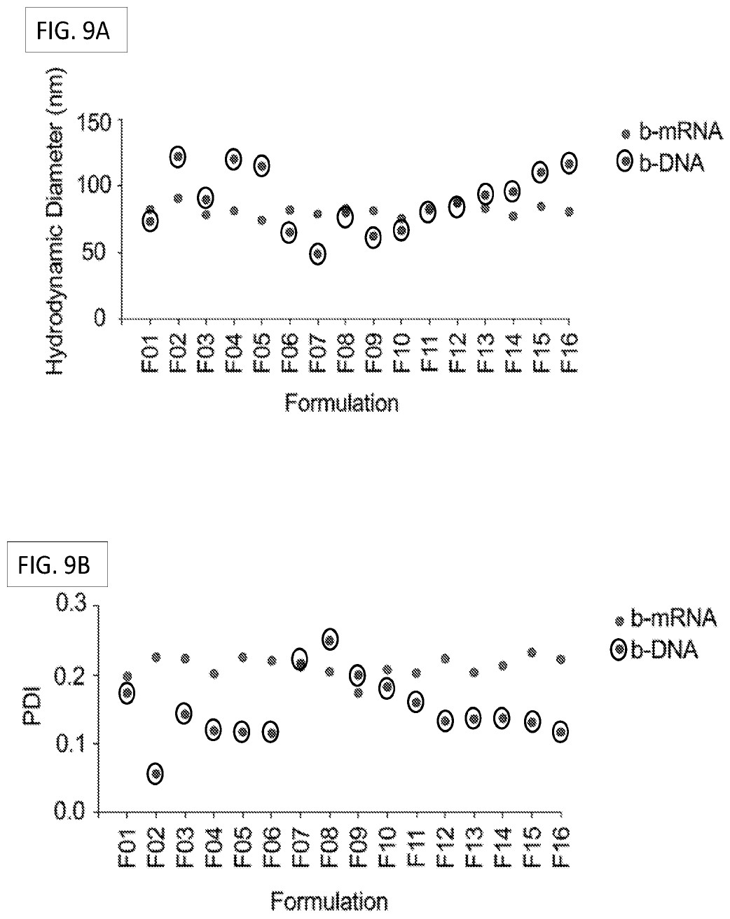

To assess this, LNPs used for b-mRNA delivery were also formulated with b-DNA used in a previous report³³. In brief, b-DNA included universal primer sites, a 10-nucleotide barcode region, and a 10-nucleotide UMI region to minimize PCR bias (FIG. 6A). We formulated the 16 LNPs that were used previously to encapsulate b-mRNA (Table 1), now encapsulating 16 different b-DNAs (FIG. 6A). As anticipated, switching nucleic acid cargo from b-mRNA to b-DNA in LNPs altered LNP hydrodynamic diameter and PDI for all 16 formulations (FIG. 9). To evaluate the delivery of b-DNA LNPs, all 16 b-DNA LNPs were pooled and administrated to mice intravenously. 4 hours post-injection, both the liver and spleen were isolated, and delivery of each b-DNA LNP formulation was quantified using deep sequencing in a similar manner to b-mRNA LNP delivery discussed previously (FIG. 6B and FIG. 6C).

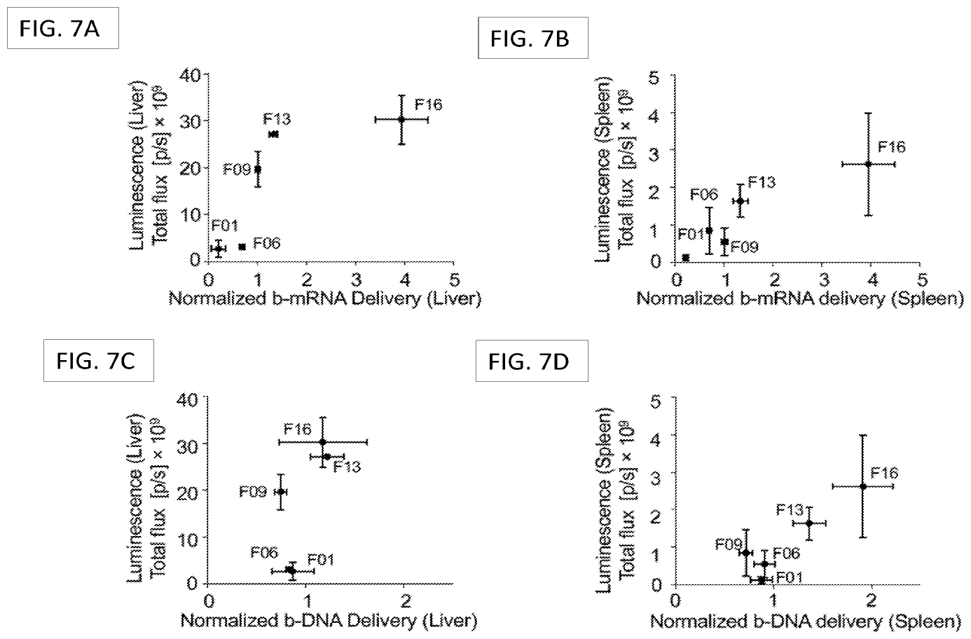

When b-DNA was encapsulated in LNPs, F04 was identified as one of the lead formulations for both liver and spleen delivery (FIG. 6B and FIG. 6C). However, when b-mRNA was encapsulated in the LNP, F04 exhibited lower delivery compared to several other formulations (FIG. 3C and FIG. 3D). In order to better understand these differences, delivery of 16 b-mRNA LNPs was plotted against the delivery of 16 b-DNA LNPs to the liver (FIG. 6D) and spleen (FIG. 6E). Weak correlations between b-DNA delivery and b-mRNA delivery to both the liver (R2=0.0164) and spleen (R2=0.2505) were found based off a linear regression model, demonstrating that differences in nucleic acid cargo can alter LNP delivery in vivo. This result was supported in recent studies, as altering LNP nucleic acid cargo has been shown to dramatically impact LNP physical characteristics³⁶. For instance, the location of all components (ionizable lipid, excipient, and nucleic acid) within LNPs may affect the amount of PEG chains exposed on the LNP surface, thus affecting LNP interactions with serum proteins and ultimately altering LNP delivery^{29, 36, 52.} The use of b-DNA has previously enabled rapid, high-throughput in vivo screening of LNPs for small nucleic acid delivery, such as siRNA and sgRNA^{35, 53}. However, predicting the functionality of a therapeutic mRNA using LNPs containing small nucleic acid has potential challenges. One potential challenge is that b-DNA is relatively similar in length to siRNA and sgRNA but is orders of magnitude smaller than mRNA. Therefore, an alteration in cargo from a shorter b-DNA to a longer mRNA can alter the fundamental structure and physical properties of the LNP formulation³⁶. By contrast, b-mRNA by design has a similar size to a therapeutic mRNA, and therefore may minimize changes in LNP physical properties and ultimately delivery. To assess this, we directly compared the predictability of Trilink-mRNA LNP delivery to either b-mRNA LNP delivery or b-DNA LNP delivery, for both the liver (FIG. 7A and FIG. 7C) and spleen (FIG. 7B and FIG. 7D). By plotting b-mRNA delivery versus mRNA transfection in vivo, we found a strong correlation for the b-mRNA system, where higher b-mRNA delivery generally corresponded to higher mRNA transfection (FIG. 7A and FIG. 7B). By contrast, the correlation was less clear for the b-DNA system by plotting b-DNA delivery versus mRNA transfection (FIGS. 7C and 7D). Therefore, our b-mRNA system, either alone or in combination with other novel systems that co-deliver b-DNA with b-mRNA54, may provide an accurate means for accelerated in vivo mRNA delivery screening.

In this study, we demonstrated that b-mRNA LNPs are a potential high-throughput tool for tracking tissue-specific delivery of a functional mRNA. Furthermore, our studies comparing b-mRNA LNPs and b-DNA LNPs indicated that the structure of different nucleic acid cargo (i.e., b-DNA versus b-mRNA) can affect LNP physical properties and subsequently alter their in vivo delivery. Therefore, the inclusion of a nucleic acid barcode that is similar size and structure to the therapeutic cargo is a potentially important factor for predicting therapeutic mRNA delivery. Since b-mRNA has a similar structure to functional mRNA, b-mRNA may provide an optimal “first-pass” delivery screen to identify lead formulations for mRNA delivery. The flexible design of b-mRNA allows them to be utilized as proxies for many other mRNA sequences with different sizes, such as Cas9 mRNA (4,521 nucleotides), or the smaller human erythropoietin (EPO) mRNA (858 nucleotides).

### Example 2: Materials and Methods

Barcoded templates for in vitro transcription (IVT) were constructed via PCR from a plasmid, pGL4.10[luc2] (Promega, E1751) using the following primers:

Luc_T7_F2:
(SEQ ID NO: 1)
5′-TAATACGACTCACTATAgggCATTCCGGTACTGTTGG
Luc_BC_R(N):
(SEQ ID NO: 2)
5′-GCCCAGTCATAGCCGAATAGNNNNNNNNNN
[Barcode]CCGCCCCGACTCTAGAATTA

A full list of IVT templates can be found in Table 4. All oligonucleotides were purchased from Integrated DNA Technologies with standard desalting. PCR was conducted using 1× Phusion HF buffer containing a final concentration 0.5 μM Miseq primer (Table 3), 200 μM dNTPs, and 0.4 U Phusion High-Fidelity DNA Polymerase (New England BioLabs, M0530S). The samples were denatured at 98° C. for 30 seconds then run for 35 cycles through the following conditions: 98° C. for 10 seconds, 65° C. for 30 seconds, and 72° C. for 2 minutes. This was followed by a final 10-minute extension at 72° C. Templates were segregated using 1% agarose gel (Universal Medical, IB70060), and 1.7 kb products were excised and purified via Zymoclean Gel DNA Recovery Kit (Zymo Research D4007) following the manufacturer's protocol.

TABLE 3
Library of Primer Sequences

| Primers | Sequences | SEQ ID NO |
|---|---|---|
| Miseq primer 1 | CAAGCAGAAGACGGCATACGAGATCCTGGTAGGTGACTGGAGTTCAGACGTGTG | 3 |
| Miseq primer 2 | CAAGCAGAAGACGGCATACGAGATTAAGCATGGTGACTGGAGTTCAGACGTGTG | 4 |
| Miseq primer 3 | CAAGCAGAAGACGGCATACGAGATAGATGTGCGTGACTGGAGTTCAGACGTGTG | 5 |
| Miseq primer 4 | CAAGCAGAAGACGGCATACGAGATGTCGAGCAGTGACTGGAGTTCAGACGTGTG | 6 |
| Miseq primer 5 | CAAGCAGAAGACGGCATACGAGATGAATTGCTGTGACTGGAGTTCAGACGTGTG | 7 |
| Miseq primer 6 | CAAGCAGAAGACGGCATACGAGATAAGCAACTGTGACTGGAGTTCAGACGTGTG | 8 |
| Miseq primer 7 | CAAGCAGAAGACGGCATACGAGATCTAACTGGGTGACTGGAGTTCAGACGTGTG | 9 |
| Miseq primer 8 | CAAGCAGAAGACGGCATACGAGATAGGCTCAAGTGACTGGAGTTCAGACGTGTG | 10 |
| Miseq primer 9 | CAAGCAGAAGACGGCATACGAGATCAGTTGGTGTGACTGGAGTTCAGACGTGTG | 11 |
| Miseq primer 10 | CAAGCAGAAGACGGCATACGAGATTCTGGACCGTGACTGGAGTTCAGACGTGTG | 12 |
| Miseq primer 11 | CAAGCAGAAGACGGCATACGAGATTGTTATACGTGACTGGAGTTCAGACGTGTG | 13 |
| Miseq primer 12 | CAAGCAGAAGACGGCATACGAGATTCAGCGAAGTGACTGGAGTTCAGACGTGTG | 14 |
| Miseq primer 13 | CAAGCAGAAGACGGCATACGAGATGTCAAGTTGTGACTGGAGTTCAGACGTGTG | 15 |
| Miseq primer 14 | CAAGCAGAAGACGGCATACGAGATAGGATGTGGTGACTGGAGTTCAGACGTGTG | 16 |
| Miseq primer 15 | CAAGCAGAAGACGGCATACGAGATCATTCCGAGTGACTGGAGTTCAGACGTGTG | 17 |
| Miseq primer 16 | CAAGCAGAAGACGGCATACGAGATACATCCTTGTGACTGGAGTTCAGACGTGTG | 18 |
| Miseq primer 17 | CAAGCAGAAGACGGCATACGAGATTCGTGTGCGTGACTGGAGTTCAGACGTGTG | 19 |
| Miseq primer 18 | CAAGCAGAAGACGGCATACGAGATTCGCCAGAGTGACTGGAGTTCAGACGTGTG | 20 |
| Miseq primer 19 | CAAGCAGAAGACGGCATACGAGATTCGCTATGGTGACTGGAGTTCAGACGTGTG | 21 |
| Miseq primer 20 | CAAGCAGAAGACGGCATACGAGATGGCTCCTGGTGACTGGAGTTCAGACGTGTG | 22 |
| Miseq primer 21 | CAAGCAGAAGACGGCATACGAGATATCCGACAGTGACTGGAGTTCAGACGTGTG | 23 |
| Miseq primer 22 | CAAGCAGAAGACGGCATACGAGATAACATAATGTGACTGGAGTTCAGACGTGTG | 24 |
| Miseq primer 23 | CAAGCAGAAGACGGCATACGAGATATGGTAGGGTGACTGGAGTTCAGACGTGTG | 25 |
| Miseq primer 24 | CAAGCAGAAGACGGCATACGAGATGCTAAGTAGTGACTGGAGTTCAGACGTGTG | 26 |
| Miseq primer 25 | CAAGCAGAAGACGGCATACGAGATACTTCTTCGTGACTGGAGTTCAGACGTGTG | Z1 |
| Miseq primer 26 | CAAGCAGAAGACGGCATACGAGATTAGATCCTGTGACTGGAGTTCAGACGTGTG | 28 |
| Miseq primer 27 | CAAGCAGAAGACGGCATACGAGATTTACTGTCGTGACTGGAGTTCAGACGTGTG | 29 |
| Miseq primer 28 | CAAGCAGAAGACGGCATACGAGATGGCATAGGGTGACTGGAGTTCAGACGTGTG | 30 |
| Miseq primer 29 | CAAGCAGAAGACGGCATACGAGATCAAGGCGAGTGACTGGAGTTCAGACGTGTG | 31 |
| Miseq primer 30 | CAAGCAGAAGACGGCATACGAGATGACGCTATGTGACTGGAGTTCAGACGTGTG | 32 |
| Miseq primer 31 | CAAGCAGAAGACGGCATACGAGATAAGGCGACGTGACTGGAGTTCAGACGTGTG | 33 |
| Miseq primer 32 | CAAGCAGAAGACGGCATACGAGATCCTAGAATGTGACTGGAGTTCAGACGTGTG | 34 |
| Miseq primer 33 | CAAGCAGAAGACGGCATACGAGATTGGTAACGGTGACTGGAGTTCAGACGTGTG | 35 |
| Miseq primer 34 | CAAGCAGAAGACGGCATACGAGATCATCAGACGTGACTGGAGTTCAGACGTGTG | 36 |
| Miseq primer 35 | CAAGCAGAAGACGGCATACGAGATGTGCGTAAGTGACTGGAGTTCAGACGTGTG | 37 |
| Miseq primer 36 | CAAGCAGAAGACGGCATACGAGATCTATTCAAGTGACTGGAGTTCAGACGTGTG | 38 |
| Miseq primer 37 | CAAGCAGAAGACGGCATACGAGATAGTGTCTTGTGACTGGAGTTCAGACGTGTG | 39 |
| Miseq primer 38 | CAAGCAGAAGACGGCATACGAGATCCTTGCTGGTGACTGGAGTTCAGACGTGTG | 40 |
| Miseq primer 39 | CAAGCAGAAGACGGCATACGAGATTTGCTGGAGTGACTGGAGTTCAGACGTGTG | 41 |
| Miseq primer 40 | CAAGCAGAAGACGGCATACGAGATAGCTCTGGGTGACTGGAGTTCAGACGTGTG | 42 |
| Miseq primer 41 | CAAGCAGAAGACGGCATACGAGATACCAAGGAGTGACTGGAGTTCAGACGTGTG | 43 |
| Miseq primer 42 | CAAGCAGAAGACGGCATACGAGATGATAACCTGTGACTGGAGTTCAGACGTGTG | 44 |
| Miseq primer 43 | CAAGCAGAAGACGGCATACGAGATTAGATGACGTGACTGGAGTTCAGACGTGTG | 45 |
| Miseq primer 44 | CAAGCAGAAGACGGCATACGAGATTGCGAAGGGTGACTGGAGTTCAGACGTGTG | 46 |
| Miseq primer 45 | CAAGCAGAAGACGGCATACGAGATGACCGAGAGTGACTGGAGTTCAGACGTGTG | 47 |
| Miseq primer 46 | CAAGCAGAAGACGGCATACGAGATCAGACAATGTGACTGGAGTTCAGACGTGTG | 48 |
| Miseq primer 47 | CAAGCAGAAGACGGCATACGAGATCTAGGTTCGTGACTGGAGTTCAGACGTGTG | 49 |
| Miseq primer 48 | CAAGCAGAAGACGGCATACGAGATGTTCATTAGTGACTGGAGTTCAGACGTGTG | 50 |

TABLE 4
Library of IVT Templates (where N is any base, and NNNNNNNNNN is
the barcode)

| Barcode | Sequence | SEQ ID NO |
|---|---|---|
| b-mRNA 1 | GCCCAGTCATAGCCGAATAG NNNNNNNNNN AAGTACCCTCCCGCCCCGACTCTAGAATTA | 51 |
| b-mRNA 2 | GCCCAGTCATAGCCGAATAG NNNNNNNNNN GGCAATTGTCCCGCCCCGACTCTAGAATTA | 52 |
| b-mRNA 3 | GCCCAGTCATAGCCGAATAG NNNNNNNNNN AGGTGCGTTACCGCCCCGACTCTAGAATTA | 53 |
| b-mRNA 4 | GCCCAGTCATAGCCGAATAG NNNNNNNNNN CGACGATCATCCGCCCCGACTCTAGAATTA | 54 |
| b-mRNA 5 | GCCCAGTCATAGCCGAATAG NNNNNNNNNN ATGGGAGACACCGCCCCGACTCTAGAATTA | 55 |
| b-mRNA 6 | GCCCAGTCATAGCCGAATAG NNNNNNNNNN CTGTTTCTCCCCGCCCCGACTCTAGAATTA | 56 |
| b-mRNA 7 | GCCCAGTCATAGCCGAATAG NNNNNNNNNN CGTTTGTACGCCGCCCCGACTCTAGAATTA | 57 |
| b-mRNA 8 | GCCCAGTCATAGCCGAATAG NNNNNNNNNN CCCACAAATCCCGCCCCGACTCTAGAATTA | 58 |
| b-mRNA 9 | GCCCAGTCATAGCCGAATAG NNNNNNNNNN AAGGCTGCAACCGCCCCGACTCTAGAATTA | 59 |
| b-mRNA 10 | GCCCAGTCATAGCCGAATAG NNNNNNNNNN GTCAGCATTCCCGCCCCGACTCTAGAATTA | 60 |
| b-mRNA 11 | GCCCAGTCATAGCCGAATAG NNNNNNNNNN CCTCATGGATCCGCCCCGACTCTAGAATTA | 61 |
| b-mRNA 12 | GCCCAGTCATAGCCGAATAG NNNNNNNNNN CATCGTGGAACCGCCCCGACTCTAGAATTA | 62 |
| b-mRNA 13 | GCCCAGTCATAGCCGAATAG NNNNNNNNNN AATTCCCAGCCCGCCCCGACTCTAGAATTA | 63 |
| b-mRNA 14 | GCCCAGTCATAGCCGAATAG NNNNNNNNNN CGTCGTTTTGCCGCCCCGACTCTAGAATTA | 64 |
| b-mRNA 15 | GCCCAGTCATAGCCGAATAG NNNNNNNNNN AAAGGCGAGACCGCCCCGACTCTAGAATTA | 65 |
| b-mRNA 16 | GCCCAGTCATAGCCGAATAG NNNNNNNNNN CTCTGATCTGCCGCCCCGACTCTAGAATTA | 66 |
| b-mRNA 17 | GCCCAGTCATAGCCGAATAG NNNNNNNNNN AGAACGTGTCCCGCCCCGACTCTAGAATTA | 67 |
| b-mRNA 18 | GCCCAGTCATAGCCGAATAG NNNNNNNNNN AGACCTTAGCCCGCCCCGACTCTAGAATTA | 68 |
| b-mRNA 19 | GCCCAGTCATAGCCGAATAG NNNNNNNNNN AGGTCGAACACCGCCCCGACTCTAGAATTA | 69 |
| b-mRNA 20 | GCCCAGTCATAGCCGAATAG NNNNNNNNNN TGTCTTTGGGCCGCCCCGACTCTAGAATTA | 70 |
| b-mRNA 21 | GCCCAGTCATAGCCGAATAG NNNNNNNNNN TTCCTACCTGCCGCCCCGACTCTAGAATTA | 71 |
| b-mRNA 22 | GCCCAGTCATAGCCGAATAG NNNNNNNNNN CGCGATAATGCCGCCCCGACTCTAGAATTA | 72 |
| b-mRNA 23 | GCCCAGTCATAGCCGAATAG NNNNNNNNNN TCAGTCTCTGCCGCCCCGACTCTAGAATTA | 73 |
| b-mRNA 24 | GCCCAGTCATAGCCGAATAG NNNNNNNNNN GTGCATGTCACCGCCCCGACTCTAGAATTA | 74 |
| b-mRNA 25 | GCCCAGTCATAGCCGAATAG NNNNNNNNNN GGAACCAACACCGCCCCGACTCTAGAATTA | 75 |
| b-mRNA 26 | GCCCAGTCATAGCCGAATAG NNNNNNNNNN GTTCAGAGAGCCGCCCCGACTCTAGAATTA | 76 |
| b-mRNA 27 | GCCCAGTCATAGCCGAATAG NNNNNNNNNN TTGGCTAGTGCCGCCCCGACTCTAGAATTA | 77 |
| b-mRNA 28 | GCCCAGTCATAGCCGAATAG NNNNNNNNNN GGCAAAGTCACCGCCCCGACTCTAGAATTA | 78 |
| b-mRNA 29 | GCCCAGTCATAGCCGAATAG NNNNNNNNNN CTTCGCTGAACCGCCCCGACTCTAGAATTA | 79 |
| b-mRNA 30 | GCCCAGTCATAGCCGAATAG NNNNNNNNNN GTATGCCTGTCCGCCCCGACTCTAGAATTA | 80 |
| b-mRNA 31 | GCCCAGTCATAGCCGAATAG NNNNNNNNNN TTAGGAGTCGCCGCCCCGACTCTAGAATTA | 81 |
| b-mRNA 32 | GCCCAGTCATAGCCGAATAG NNNNNNNNNN AGATAGCGTGCCGCCCCGACTCTAGAATTA | 82 |
| b-mRNA 33 | GCCCAGTCATAGCCGAATAG NNNNNNNNNN AAGCGGACTTCCGCCCCGACTCTAGAATTA | 83 |
| b-mRNA 34 | GCCCAGTCATAGCCGAATAG NNNNNNNNNN CGGTCTAGATCCGCCCCGACTCTAGAATTA | 84 |
| b-mRNA 35 | GCCCAGTCATAGCCGAATAG NNNNNNNNNN AAGGGGGATTCCGCCCCGACTCTAGAATTA | 85 |
| b-mRNA 36 | GCCCAGTCATAGCCGAATAG NNNNNNNNNN CAGATGCGAACCGCCCCGACTCTAGAATTA | 86 |
| b-mRNA 37 | GCCCAGTCATAGCCGAATAG NNNNNNNNNN GGAACTCCAACCGCCCCGACTCTAGAATTA | 87 |
| b-mRNA 38 | GCCCAGTCATAGCCGAATAG NNNNNNNNNN GATTGGCCAACCGCCCCGACTCTAGAATTA | 88 |
| b-mRNA 39 | GCCCAGTCATAGCCGAATAG NNNNNNNNNN TGTCGCCTTTCCGCCCCGACTCTAGAATTA | 89 |
| b-mRNA 40 | GCCCAGTCATAGCCGAATAG NNNNNNNNNN CAGCGACATTCCGCCCCGACTCTAGAATTA | 90 |
| b-mRNA 41 | GCCCAGTCATAGCCGAATAG NNNNNNNNNN CCATACGTTGCCGCCCCGACTCTAGAATTA | 91 |
| b-mRNA 42 | GCCCAGTCATAGCCGAATAG NNNNNNNNNN GGTTAGGTAGCCGCCCCGACTCTAGAATTA | 92 |
| b-mRNA 43 | GCCCAGTCATAGCCGAATAG NNNNNNNNNN TACACCGTGACCGCCCCGACTCTAGAATTA | 93 |
| b-mRNA 44 | GCCCAGTCATAGCCGAATAG NNNNNNNNNN TACTTCTGGGCCGCCCCGACTCTAGAATTA | 94 |
| b-mRNA 45 | GCCCAGTCATAGCCGAATAG NNNNNNNNNN AAGCGCTTGTCCGCCCCGACTCTAGAATTA | 95 |
| b-mRNA 46 | GCCCAGTCATAGCCGAATAG NNNNNNNNNN CGGTTAATCCCCGCCCCGACTCTAGAATTA | 96 |
| b-mRNA 47 | GCCCAGTCATAGCCGAATAG NNNNNNNNNN TATCACGAGGCCGCCCCGACTCTAGAATTA | 97 |
| b-mRNA 48 | GCCCAGTCATAGCCGAATAG NNNNNNNNNN CCGAATAGTCCCGCCCCGACTCTAGAATTA | 98 |
| Luc_T7_F2 | TAATACGACTCACTATAggg CATTCCGGTACTGTTGG | 99 |
| Luc_Seq_US1 | AGACGTGTGCTCTTCCGATCT GGACGAGGTGCCTAAAGGAC | 100 |
| NeoR_Seq_US2 | ACACGACGCTCTTCCGATCT GCCCAGTCATAGCCGAATAG | 101 |

IVT

Uncapped RNA was synthesized via IVT using a modified HiScribe T7 High Yield RNA Synthesis Kit (New England Biolabs, E2040S) containing 100 ng of purified template in 20 μL reactions. The manufacturer's protocol was modified by replacing CTP (cytidine-5′-triphosphate) with 5mCTP (Trilink biotechnologies, N-1014) in an overnight incubation at 37° C. DNA was degraded with 2 U of RQ1 DNase (Promega, M6101) for 30 minutes at 37° C. RNA was purified using a RNeasy MinElute Cleanup Kit (Qiagen, 74204) following the manufacturer's protocol, eluting with 50 μL RNase-free H2O. For different mRNA modifications, chemically modified nucleotides were completely substituted for their unmodified counterparts while synthesizing the mRNA.

RNA Capping and Tailing

25 μg RNA was resuspended in 15 μL RNase-free H₂O and denatured at 65° C. for 5 minutes, and immediately placed on ice. RNA was capped using the Vaccinia Capping System (New England BioLabs, M2080S) in 50 μL reaction following the manufacturer's protocol and incubated at 37° C. for 30 minutes. Poly(A) tails were added using E. coli Poly(A) Polymerase (New England BioLabs, M0276S) by adding 10 μL 10×PAP Reaction Buffer, 10 μL 10 mM ATP, 5 μL (25 U) E. Coli PAP, and 25 μL RNase-free H₂O and incubated at 37° C. for 30 minutes. Reactions were stopped with the addition of 100 μL of RNA binding buffer (Zymo Research, R1013-2-25). mRNA was purified using a Zymo RNA Clean & Concentrator Kit (Zymo Research, R1018) following the manufacturer's protocol. Quality control testing of mRNA was conducted using a Bioanalyzer (Agilent 2100 Bioanalyzer; Agilent Technologies)

RNA Extraction and cDNA Synthesis

30 mg of disrupted frozen tissue was resuspended in TRIzol™ Reagent (Thermo Fisher Scientific, 15596026); total RNA was extracted following the manufacturer's protocol. 2 μg of extracted RNA was treated with 1U RQ1 DNase, 1×RQ1 DNase buffer, and 20 U RNase inhibitor (New England Biolabs, M0314S) for 30 minutes at 37° C. to remove any remaining DNA in solution. The reaction was terminated by adding 1 μL Stop solution and incubated for 10 minutes at 65° C. 1 μL Oligo dT (Integrated DNA Technologies, 51-01-15-05) was added to each reaction and denatured for 5 minutes at 70° C., and then immediately placed on ice. Reverse transcription of the DNase-treated RNA was carried out in a 20 μL reaction using 1 μL of GoScript Reverse Transcriptase (Promega, A5003) containing a final concentration of 1×GoScript Reaction Buffer, 2.5 mM MgCl2, 0.5 mM dNTPs using the following cycling conditions: 25° C. for 5 minutes, 42° C. for 1 hour, and 70° C. for 15 minutes.

Barcoded mRNA (b-mRNA) Library Preparation

Library templates were prepared via two stages of PCR. In the first stage, adapters were added to the cDNA using the following primers:

Luc_Seq_US1:
(SEQ ID NO: 100)
5′-AGACGTGTGCTCTTCCGATCTGGACGAGGTGCCTAAAGGAC
NeoR_Seq_US2:
(SEQ ID NO: 101)
5′-ACACGACGCTCTTCCGATCTGCCCAGTCATAGCCGAATAG

PCR was carried out in 1× Phusion HF buffer containing a final concentration of 0.5 μM

Luc Seq US1, 0.5 μM NeoR_Seq_US2, 200 μM dNTPs, and 0.4 U Phusion High-Fidelity

DNA Polymerase. Templates were denatured at 98° C. for 30 seconds followed by 16 cycles of: 98° C. for 10 seconds, 65° C. for 30 seconds, 72° C. for 2 minutes followed by a final 10-minute extension at 72° C. with an expected product size of 218 bp. Templates were purified using 1.8 volumes of Mag-Bind TotalPure NGS beads (Omega Biotek, M1378-00), followed by two 80% ethanol washes and elution in 20 μL TE.

In the second stage, Illumina primers were added to the cDNA using the following primers from a previous study⁵¹:

Forward (Index-Base):
(SEQ ID NO: 102)
5′-AATGATACGGCGACCACCGAGATCTACACTCTTTCCCTACACGACG
CTCTTCCGAT CT
Reverse (Universal):
(SEQ ID NO: 103)
5′-TGACTGGAGTTCAGACGTGTGCTCTTCCGATCT
Miseq primers (Table 3):
(SEQ ID Nos: 3-50)
5′-CAAGCAGAAGACGGCATACGAGAT[index]
GTGACTGGAGTTCAGACGTGTGCTCT TCCGATCT

cDNA was denatured at 98° C. for 30 seconds followed by 16 cycles of 98° C. for 10 seconds, 65° C. for 30 seconds, 72° C. for 2 minutes followed by a final 10-minute extension at 72° C. with an expected size of 301 bp. PCR products were purified using a 1.8×volume ratio of Mag-Bind TotalPure NGS beads (Omega Biotek, M1378-00), followed by two 80% Ethanol washes and eluted in 20 μL TE. The purified products were kept frozen until deep sequencing.

b-DNA Library Preparation

b-DNA design parameters were adopted from a previous report⁵¹. b-DNA consisted of 61 nucleotide single-stranded DNA with three consecutive phosphorothioate bonds at each end. The barcode region was composed of 10 nucleotides in the center of the oligonucleotide. An additional 10 random nucleotides were included at 3′ of the barcode region. The 5′ and 3′ ends of each b-DNA contained priming sites for Illumina adapters. A full list of b-DNA sequences can be found in Table 5. Desalted oligonucleotides were ordered from Integrated DNA Technologies. To extract DNA from a tissue sample, approximately 30 mg of disrupted frozen tissue was resuspended in lysis buffer⁵⁵ that contained 100 mM Tris-HCl (Fisher Scientific, 50155887), 5 mM EDTA (Fisher Scientific, 20 50997738), 0.2% SDS (Fisher Scientific, 507513793), 200 mM NaCl (Fisher Scientific, S318100), and 0.2 mg/mL proteinase K (Thermo Fisher Scientific, PI17916). Extracted DNA was further purified by Zymo Oligo Clean and Concentrator columns (Zymo Research, D4060) according to the manufacturer's instructions. b-DNA amplification was conducted by PCR using the following recipe: 5 μL 5x HF Phusion buffer, 0.5 μL 10 mM dNTPs, 0.25 μL Phusion High-Fidelity DNA Polymerase (Thermo Fisher Scientific, F530S), 1.18 μL extracted DNA, 0.5 μL 5 μM reverse (universal), 0.5 μL 5 μM Miseq primer (Table 3), 0.5 μL 0.5 μM forward (Index-base), 2 μL DMSO, and 15.25 μL H2O. PCR cycling conditions were 98° C. for 12 seconds, 67° C. for 22 seconds, and 72° C. for 28 seconds, for a total of 35 cycles.

TABLE 5
bDNA sequences (where N is any base, and NNNNNNNNNN is the
barcode)

| | | SEQ ID |
|---|---|---|
| Barcode | Sequences | NO |
| b-DNA 1 | A*G*A*CGTGTGCTCTTCCGATCT GAGGGTACTT NNNNNNNNNN AGATCGGAAGAGCGTCG*T*G*T | 104 |
| b-DNA 2 | A*G*A*CGTGTGCTCTTCCGATCT GACAATTGCC NNNNNNNNNN AGATCGGAAGAGCGTCG*T*G*T | 105 |
| b-DNA 3 | A*G*A*CGTGTGCTCTTCCGATCT TAACGCACCT NNNNNNNNNN AGATCGGAAGAGCGTCG*T*G*T | 106 |
| b-DNA 4 | A*G*A*CGTGTGCTCTTCCGATCT ATGATCGTCG NNNNNNNNNN AGATCGGAAGAGCGTCG*T*G*T | 107 |
| b-DNA 5 | A*G*A*CGTGTGCTCTTCCGATCT TGTCTCCCAT NNNNNNNNNN AGATCGGAAGAGCGTCG*T*G*T | 108 |
| b-DNA 6 | A*G*A*CGTGTGCTCTTCCGATCT GGAGAAACAG NNNNNNNNNN AGATCGGAAGAGCGTCG*T*G*T | 109 |
| b-DNA 7 | A*G*A*CGTGTGCTCTTCCGATCT CGTACAAACG NNNNNNNNNN AGATCGGAAGAGCGTCG*T*G*T | 110 |
| b-DNA 8 | A*G*A*CGTGTGCTCTTCCGATCT GATTTGTGGG NNNNNNNNNN AGATCGGAAGAGCGTCG*T*G*T | 111 |
| b-DNA 9 | A*G*A*CGTGTGCTCTTCCGATCT TTGCAGCCTT NNNNNNNNNN AGATCGGAAGAGCGTCG*T*G*T | 112 |
| b-DNA 10 | A*G*A*CGTGTGCTCTTCCGATCT GAATGCTGAC NNNNNNNNNN AGATCGGAAGAGCGTCG*T*G*T | 113 |
| b-DNA 11 | A*G*A*CGTGTGCTCTTCCGATCT ATCCATGAGG NNNNNNNNNN AGATCGGAAGAGCGTCG*T*G*T | 114 |
| b-DNA 12 | A*G*A*CGTGTGCTCTTCCGATCT TTCCACGATG NNNNNNNNNN AGATCGGAAGAGCGTCG*T*G*T | 115 |
| b-DNA 13 | A*G*A*CGTGTGCTCTTCCGATCT GCTGGGAATT NNNNNNNNNN AGATCGGAAGAGCGTCG*T*G*T | 116 |
| b-DNA 14 | A*G*A*CGTGTGCTCTTCCGATCT CAAAACGACG NNNNNNNNNN AGATCGGAAGAGCGTCG*T*G*T | 117 |
| b-DNA 15 | A*G*A*CGTGTGCTCTTCCGATCT TCTCGCCTTT NNNNNNNNNN AGATCGGAAGAGCGTCG*T*G*T | 118 |
| b-DNA 16 | A*G*A*CGTGTGCTCTTCCGATCT CAGATCAGAG NNNNNNNNNN AGATCGGAAGAGCGTCG*T*G*T | 119 |
| b-DNA 17 | A*G*A*CGTGTGCTCTTCCGATCT GACACGTTCT NNNNNNNNNN AGATCGGAAGAGCGTCG*T*G*T | 120 |
| b-DNA 18 | A*G*A*CGTGTGCTCTTCCGATCT GCTAAGGTCT NNNNNNNNNN AGATCGGAAGAGCGTCG*T*G*T | 121 |
| b-DNA 19 | A*G*A*CGTGTGCTCTTCCGATCT TGTTCGACCT NNNNNNNNNN AGATCGGAAGAGCGTCG*T*G*T | 122 |
| b-DNA 20 | A*G*A*CGTGTGCTCTTCCGATCT CCCAAAGACA NNNNNNNNNN AGATCGGAAGAGCGTCG*T*G*T | 123 |
| b-DNA 21 | A*G*A*CGTGTGCTCTTCCGATCT CAGGTAGGAA NNNNNNNNNN AGATCGGAAGAGCGTCG*T*G*T | 124 |
| b-DNA 22 | A*G*A*CGTGTGCTCTTCCGATCT CATTATCGCG NNNNNNNNNN AGATCGGAAGAGCGTCG*T*G*T | 125 |
| b-DNA 23 | A*G*A*CGTGTGCTCTTCCGATCT CAGAGACTGA NNNNNNNNNN AGATCGGAAGAGCGTCG*T*G*T | 126 |
| b-DNA 24 | A*G*A*CGTGTGCTCTTCCGATCT TGACATGCAC NNNNNNNNNN AGATCGGAAGAGCGTCG*T*G*T | 127 |
| b-DNA 25 | A*G*A*CGTGTGCTCTTCCGATCT TGTTGGTTCC NNNNNNNNNN AGATCGGAAGAGCGTCG*T*G*T | 128 |
| b-DNA 26 | A*G*A*CGTGTGCTCTTCCGATCT CTCTCTGAAC NNNNNNNNNN AGATCGGAAGAGCGTCG*T*G*T | 129 |
| b-DNA 27 | A*G*A*CGTGTGCTCTTCCGATCT CACTAGCCAA NNNNNNNNNN AGATCGGAAGAGCGTCG*T*G*T | 130 |
| b-DNA 28 | A*G*A*CGTGTGCTCTTCCGATCT TGACTTTGCC NNNNNNNNNN AGATCGGAAGAGCGTCG*T*G*T | 131 |
| b-DNA 29 | A*G*A*CGTGTGCTCTTCCGATCT TTCAGCGAAG NNNNNNNNNN AGATCGGAAGAGCGTCG*T*G*T | 132 |
| b-DNA 30 | A*G*A*CGTGTGCTCTTCCGATCT ACAGGCATAC NNNNNNNNNN AGATCGGAAGAGCGTCG*T*G*T | 133 |
| b-DNA 31 | A*G*A*CGTGTGCTCTTCCGATCT CGACTCCTAA NNNNNNNNNN AGATCGGAAGAGCGTCG*T*G*T | 134 |
| b-DNA 32 | A*G*A*CGTGTGCTCTTCCGATCT CACGCTATCT NNNNNNNNNN AGATCGGAAGAGCGTCG*T*G*T | 135 |
| b-DNA 33 | A*G*A*CGTGTGCTCTTCCGATCT AAGTCCGCTT NNNNNNNNNN AGATCGGAAGAGCGTCG*T*G*T | 136 |
| b-DNA 34 | A*G*A*CGTGTGCTCTTCCGATCT ATCTAGACCG NNNNNNNNNN AGATCGGAAGAGCGTCG*T*G*T | 137 |
| b-DNA 35 | A*G*A*CGTGTGCTCTTCCGATCT AATCCCCCTT NNNNNNNNNN AGATCGGAAGAGCGTCG*T*G*T | 138 |
| b-DNA 36 | A*G*A*CGTGTGCTCTTCCGATCT TTCGCATCTG NNNNNNNNNN AGATCGGAAGAGCGTCG*T*G*T | 139 |
| b-DNA 37 | A*G*A*CGTGTGCTCTTCCGATCT TTGGAGTTCC NNNNNNNNNN AGATCGGAAGAGCGTCG*T*G*T | 140 |
| b-DNA 38 | A*G*A*CGTGTGCTCTTCCGATCT TTGGCCAATC NNNNNNNNNN AGATCGGAAGAGCGTCG*T*G*T | 141 |
| b-DNA 39 | A*G*A*CGTGTGCTCTTCCGATCT AAAGGCGACA NNNNNNNNNN AGATCGGAAGAGCGTCG*T*G*T | 142 |
| b-DNA 40 | A*G*A*CGTGTGCTCTTCCGATCT AATGTCGCTG NNNNNNNNNN AGATCGGAAGAGCGTCG*T*G*T | 143 |
| b-DNA 41 | A*G*A*CGTGTGCTCTTCCGATCT CAACGTATGG NNNNNNNNNN AGATCGGAAGAGCGTCG*T*G*T | 144 |
| b-DNA 42 | A*G*A*CGTGTGCTCTTCCGATCT CTACCTAACC NNNNNNNNNN AGATCGGAAGAGCGTCG*T*G*T | 145 |
| b-DNA 43 | A*G*A*CGTGTGCTCTTCCGATCT TCACGGTGTA NNNNNNNNNN AGATCGGAAGAGCGTCG*T*G*T | 146 |
| b-DNA 44 | A*G*A*CGTGTGCTCTTCCGATCT CCCAGAAGTA NNNNNNNNNN AGATCGGAAGAGCGTCG*T*G*T | 147 |
| b-DNA 45 | A*G*A*CGTGTGCTCTTCCGATCT ACAAGCGCTT NNNNNNNNNN AGATCGGAAGAGCGTCG*T*G*T | 148 |
| b-DNA 46 | A*G*A*CGTGTGCTCTTCCGATCT GGATTAACCG NNNNNNNNNN AGATCGGAAGAGCGTCG*T*G*T | 149 |
| b-DNA 47 | A*G*A*CGTGTGCTCTTCCGATCT CCTCGTGATA NNNNNNNNNN AGATCGGAAGAGCGTCG*T*G*T | 150 |
| b-DNA 48 | A*G*A*CGTGTGCTCTTCCGATCT GACTATTCGG NNNNNNNNNN AGATCGGAAGAGCGTCG*T*G*T | 151 |

Primer sequences were shown below:

Forward (Index-Base):
(SEQ ID NO: 102)
5′-AATGATACGGCGACCACCGAGATCTACACTCTTTCCCTACACGACG
CTCTTCCGATCT
Reverse (Universal):
(SEQ ID NO: 103)
5′-TGACTGGAGTTCAGACGTGTGCTCTTCCGATCT
Miseq primers (Table 3):
(SEQ ID Nos: 3-50)
5′-CAAGCAGAAGACGGCATACGAGAT[index]
GTGACTGGAGTTCAGACGTGTGCTCTTCCGATCT

PCR products were run by gel electrophoresis on 1.4% agarose (Universal Medical, IB70060) in Tris-acetate-EDTA buffer (Fisher Scientific, 24710030). Amplified b-DNA (144 bp) was excised from the gel, pooled, and purified by Zymo Gel Extraction columns (Zymo Research, D4001) according to the manufacturer's instructions. The purified products were kept frozen until deep sequencing was performed.

Lipid Nanoparticle (LNP) Formulation

LNPs were formulated by mixing an aqueous phase containing mRNA or DNA with an ethanol phase containing ionizable lipids and excipients using a microfluidic chip device⁴⁷.

Specifically, the ethanol phase contained a mixture of an ionizable lipid (C12-200, synthesized as previously described⁵⁶), 1,2-dioleoyl-sn-glycero-3-phosphoethanolamine (DOPE, Avanti Polar Lipids, 850725P), cholesterol (Sigma-Aldrich, C8667), and 1,2-dimyristoylsn-glycero-3-phosphoethanolamine-N-[methoxy(polyethyleneglycol)-2000] (ammonium salt) (C14-PEG 2000, Avanti Polar Lipids, 880150P) at predetermined molar ratios shown in Table 1. The aqueous phase was prepared in 10 mM citrate, pH 3.0 buffer (Teknova, Q2445) with either in-house synthesized b-mRNA, Luciferase mRNA (Trilink Biotechnologies), or b-DNA (Integrated DNA Technologies). Syringe pumps were used to perfuse the ethanol and aqueous phases at a 3:1 ratio through the microfluidic device⁴⁷.

The resulting LNPs were dialyzed against PBS in a 20,000 MWCO cassette at room temperature for 2 hours and then extruded through a 0.22 μm sterile filter (Genesee Scientific, 25243).

LNP Characterization

DNA or mRNA concentration in LNP formulations was determined using a NanoDrop Spectrophotometer (Thermo Fisher Scientific). To calculate mRNA encapsulation efficiency within LNPs, a modified Quant-iT RiboGreen RNA assay (Thermo Fisher Scientific, R11490) was used as previously described²³. LNP hydrodynamic diameter and polydispersity (PDI) were measured using a Zetasizer Nano ZS machine (Malvern Instrument). For analysis of LNP structure using cryogenic-transmission electron microscopy (Cryo-TEM), LNP samples were prepared in a vitrification system (25° C., −100% humidity). A 3 μL sample of LNP solution was dropped on a lacey copper grid coated with a continuous carbon film and blotted to remove excess sample without damaging the carbon layer. A grid was mounted on a Gatan 626 single tilt cryogenic³⁷ holder equipped in the TEM column. Images of LNP samples were recorded on an UltraScan 1000 CCD camera (Gatan).

In Vitro mRNA Delivery

bEnd.3 mouse cerebral cortex endothelial cells (ATCC) were maintained 1 at 37° C. and 5% CO2 in high glucose Dulbecco's Modified Eagles Medium (Thermo Fisher) supplemented with 10% fetal bovine serum (by volume), 20 U/mL penicillin and 20 U/mL streptomycin. Cells were seeded in black 48-well plates at a density of 30,000 cells per well. After 24 hours incubation, cells were then treated with LNPs encapsulating different b-mRNA (modified with either pseudouridine (w) or 5-methylcytosine (m5C)) or commercially available Trilink-mRNA at different concentration (10 nM, 25 nM, or 50 nM). After 48 hours transfection, cells were washed with PBS and incubated with D-luciferin (150 μg/mL). Subsequently, luciferase activity was measured using an IVIS imaging system (PerkinElmer).

Animal Experiments

All procedures were performed under an animal protocol approved by the University of Pennsylvania Institutional Animal Care and Use Committee (IACUC). To evaluate b-mRNA or b-DNA delivery, 8-week-old female C57BL/6 mice (Charles River Labs, 18-21 g) were injected intravenously via the tail vein with a pool of different barcoded LNPs, at the amount of 0.25 μg b-mRNA or 1 μg b-DNA per formulation. To quantify mRNA delivery and luciferase in vivo, mice were injected intravenously via the tail vein with LNPs containing 5 μg of either mRNA coding for luciferase (Trilink Biotechnologies) or b-mRNA coding for luciferase. For all experiments, tissues were harvested 4 hours post-injection. To evaluate b-mRNA delivery or b-DNA delivery, tissues were snap-frozen in liquid nitrogen, disrupted into powder using a Geno/Grinder (SPEX Sample Prep), and stored in a −80° C. freezer. To evaluate luciferase expression, mice were administered via an intraperitoneal injection of 130 μL of D-luciferin (30 mg/mL in PBS) 15 minutes before they were sacrificed. Luminescence of harvested organs (liver, spleen, lymph node, lungs, heart, brain, pancreas and, kidneys) was analyzed using an IVIS imaging system (PerkinElmer) and quantified using Living Image Software (PerkinElmer).

Deep Sequencing and Barcode Delivery Quantification

All deep-sequencing runs were performed using multiplexed runs on Illumina MiSeq (Illumina). PCR product pools were quantitated using the KAPA Library Quantification Kit for next generation sequencing. PCR product pools were loaded onto flow cells at 4 nM concentration. Python scripts were written to quantify barcodes from Illumina fastq files.

b-mRNA delivery or b-DNA delivery of a specific barcode to a certain tissue was calculated according to the following 3 steps: (i) dividing the number of sequencing reads of one barcode delivered by a single LNP formulation by the total amount of reads from all barcodes delivered by all LNPs in a specific tissue; (ii) dividing the number of sequencing reads of the same barcode (utilized in (i)) by the total amount of reads from all barcodes of all LNPs in the non-injected LNP pool (iii) dividing the results from (i) by the results from (ii).

All publications cited in this specification are incorporated herein by reference. While the invention has been described with reference to particular embodiments, it will be appreciated that modifications can be made without departing from the spirit of the invention. Such modifications are intended to fall within the scope of the appended claims.

### REFERENCES

1. Oberli, M. A.; Reichmuth, A. M.; Dorkin, J. R.; Mitchell, M. J.; Fenton, O. S.; Jaklenec, A.; Anderson, D. G.; Langer, R.; Blankschtein, D., Lipid nanoparticle assisted mRNA delivery for potent cancer immunotherapy. Nano letters 2016, 17 (3), 1326-1335.
2. Cheng, Q.; Wei, T.; Jia, Y.; Farbiak, L.; Zhou, K.; Zhang, S.; Wei, Y.; Zhu, H.; Siegwart, D. J., Dendrimer□Based Lipid Nanoparticles Deliver Therapeutic FAH mRNA to Normalize Liver Function and Extend Survival in a Mouse Model of Hepatorenal Tyrosinemia Type I. Advanced Materials 2018, 1805308.
3. Luo, X.; Li, B.; Zhang, X.; Zhao, W.; Bratasz, A.; Deng, B.; McComb, D.; Dong, Y., Dual-functional lipid-like nanoparticles for delivery of mRNA and MRI contrast agents. Nanoscale 2017, 9 (4), 1575-1579.
4. Zhang, R.; Billingsley, M. M.; Mitchell, M. J., Biomaterials for vaccine-based cancer immunotherapy. Journal of Controlled Release 2018.
5. Riley, R. S.; June, C. H.; Langer, R.; Mitchell, M. J., Delivery technologies for cancer immunotherapy. Nature Reviews Drug Discovery 2019, 1.
6. Hajj, K. A.; Whitehead, K. A., Tools for translation: non-viral materials for therapeutic mRNA delivery. Nature Reviews Materials 2017, 2 (10), 17056.
7. Mukalel, A. J.; Riley, R. S.; Zhang, R.; Mitchell, M. J., Nanoparticles for Nucleic Acid Delivery: Applications in Cancer Immunotherapy. Cancer letters 2019.
8. Pardi, N.; Hogan, M. J.; Porter, F. W.; Weissman, D., mRNA vaccines—a new era in vaccinology. Nature Reviews Drug Discovery 2018, 17 (4), 261.
9. Kowalski, P. S.; Rudra, A.; Miao, L.; Anderson, D. G., Delivering the Messenger: Advances in Technologies for Therapeutic mRNA Delivery. Molecular Therapy 2019.
10. Bettinger, T.; Carlisle, R. C.; Read, M. L.; Ogris, M.; Seymour, L. W., Peptide mediated RNA delivery: a novel approach for enhanced transfection of primary and post-mitotic cells. Nucleic acids research 2001, 29 (18), 3882-3891.
11. Sahin, U.; Karikó, K.; Türeci, Ö., mRNA-based therapeutics—developing a new class of drugs. Nature reviews Drug discovery 2014, 13 (10), 759.
12. Islam, M. A.; Reesor, E. K.; Xu, Y.; Zope, H. R.; Zetter, B. R.; Shi, J., Biomaterials for mRNA delivery. Biomaterials science 2015, 3 (12), 1519-1533.
13. Weissman, D.; Karikó, K., mRNA: fulfilling the promise of gene therapy. Molecular Therapy 2015, 23 (9), 1416-1417.
14. Weide, B.; Carralot, J.-P.; Reese, A.; Scheel, B.; Eigentler, T. K.; Hoerr, I.; Rammensee, H.-G.; Garbe, C.; Pascolo, S., Results of the first phase I/II clinical vaccination trial with direct injection of mRNA. Journal of immunotherapy 2008, 31 (2), 180-188.
15. Weide, B.; Pascolo, S.; Scheel, B.; Derhovanessian, E.; Pflugfelder, A.; Eigentler, T. K.; Pawelec, G.; Hoerr, I.; Rammensee, H.-G.; Garbe, C., Direct injection of protamine-protected mRNA: results of a phase 1/2 vaccination trial in metastatic melanoma patients. Journal of immunotherapy 2009, 32 (5), 498-507.
16. Kranz, L. M.; Diken, M.; Haas, H.; Kreiter, S.; Loquai, C.; Reuter, K. C.; Meng, M.; Fritz, D.; Vascotto, F.; Hefesha, H., Systemic RNA delivery to dendritic cells exploits antiviral defence for cancer immunotherapy. Nature 2016, 534 (7607), 396.
17. Whitehead, K. A.; Dorkin, J. R.; Vegas, A. J.; Chang, P. H.; Veiseh, O.; Matthews, J.; Fenton, O. S.; Zhang, Y.; Olejnik, K. T.; Yesilyurt, V., Degradable lipid nanoparticles with predictable in vivo siRNA delivery activity. Nature communications 2014, 5, 4277.
18. Smith, C. E.; Zain, R., Therapeutic oligonucleotides: state of the art. Annual review of pharmacology and toxicology 2019, 59, 605-630.
19. Fenton, O. S.; Kauffman, K. J.; Kaczmarek, J. C.; McClellan, R. L.; Jhunjhunwala, S.; Tibbitt, M. W.; Zeng, M. D.; Appel, E. A.; Dorkin, J. R.; Mir, F. F., Synthesis and biological evaluation of ionizable lipid materials for the in vivo delivery of messenger RNA to B lymphocytes. Advanced Materials 2017, 29 (33), 1606944.
20. Fenton, O. S.; Kauffman, K. J.; McClellan, R. L.; Appel, E. A.; Dorkin, J. R.; Tibbitt, M. W.; Heartlein, M. W.; DeRosa, F.; Langer, R.; Anderson, D. G., Bioinspired alkenyl amino alcohol ionizable lipid materials for highly potent in vivo mRNA delivery. Advanced materials 2016, 28 (15), 2939-2943.
21. Hajj, K. A.; Ball, R. L.; Deluty, S. B.; Singh, S. R.; Strelkova, D.; Knapp, C. M.; Whitehead, K. A., Branched□Tail Lipid Nanoparticles Potently Deliver mRNA In Vivo due to Enhanced Ionization at Endosomal pH. Small 2019, 1805097.
22. Dahlman, J. E.; Barnes, C.; Khan, O. F.; Thiriot, A.; Jhunjunwala, S.; Shaw, T. E.; Xing, Y.; Sager, H. B.; Sahay, G.; Speciner, L., In vivo endothelial siRNA delivery using polymeric nanoparticles with low molecular weight. Nature nanotechnology 2014, 9 (8), 648.
23. Dong, Y.; Love, K. T.; Dorkin, J. R.; Sirirungruang, S.; Zhang, Y.; Chen, D.; Bogorad, R. L.; Yin, H.; Chen, Y.; Vegas, A. J., Lipopeptide nanoparticles for potent and selective siRNA delivery in rodents and nonhuman primates. Proceedings of the National Academy of Sciences 2014, 111 (11), 3955-3960.
24. Akinc, A.; Zumbuehl, A.; Goldberg, M.; Leshchiner, E. S.; Busini, V.; Hossain, N.; Bacallado, S. A.; Nguyen, D. N.; Fuller, J.; Alvarez, R., A combinatorial library of lipid like materials for delivery of RNAi therapeutics. Nature biotechnology 2008, 26 (5), 561.
25. Cheng, Q.; Wei, T.; Jia, Y.; Farbiak, L.; Zhou, K.; Zhang, S.; Wei, Y.; Zhu, H.; Siegwart, D. J., Dendrimer□Based Lipid Nanoparticles Deliver Therapeutic FAH mRNA to Normalize Liver Function and Extend Survival in a Mouse Model of Hepatorenal Tyrosinemia Type I. Advanced Materials 2018, 30 (52), 1805308.
26. Siegwart, D. J.; Whitehead, K. A.; Nuhn, L.; Sahay, G.; Cheng, H.; Jiang, S.; Ma, M.; Lytton-Jean, A.; Vegas, A.; Fenton, P., Combinatorial synthesis of chemically diverse core-shell nanoparticles for intracellular delivery. Proceedings of the National Academy of Sciences 2011, 108 (32), 12996-13001.
27. Granot, Y.; Peer, D. In Delivering the right message: Challenges and opportunities in lipid nanoparticles-mediated modified mRNA therapeutics—An innate immune system standpoint, Seminars in immunology, Elsevier: 2017; pp 68-77.
28. Varkouhi, A. K.; Scholte, M.; Storm, G.; Haisma, H. J., Endosomal escape pathways for delivery of biologicals. Journal of Controlled Release 2011, 151 (3), 220-228.
29. Mui, B. L.; Tam, Y. K.; Jayaraman, M.; Ansell, S. M.; Du, X.; Tam, Y. Y. C.; Lin, P. J.; Chen, S.; Narayanannair, J. K.; Rajeev, K. G., Influence of polyethylene glycol lipid desorption rates on pharmacokinetics and pharmacodynamics of siRNA lipid nanoparticles. Molecular Therapy-Nucleic Acids 2013, 2, e139.
30. Wilhelm, S.; Tavares, A. J.; Dai, Q.; Ohta, S.; Audet, J.; Dvorak, H. F.; Chan, W. C., Analysis of nanoparticle delivery to tumours. Nature reviews materials 2016, 1 (5), 16014.
31. Yang, Y.-S. S.; Atukorale, P. U.; Moynihan, K. D.; Bekdemir, A.; Rakhra, K.; Tang, L.; Stellacci, F.; Irvine, D. J., High-throughput quantitation of inorganic nanoparticle biodistribution at the single-cell level using mass cytometry. Nature communications 2017, 8, 14069.
32. Yaari, Z.; Da Silva, D.; Zinger, A.; Goldman, E.; Kajal, A.; Tshuva, R.; Barak, E.; Dahan, N.; Hershkovitz, D.; Goldfeder, M., Theranostic barcoded nanoparticles for personalized cancer medicine. Nature communications 2016, 7, 13325.
33. Dahlman, J. E.; Kauffman, K. J.; Xing, Y.; Shaw, T. E.; Mir, F. F.; Dlott, C. C.; Langer, R.; Anderson, D. G.; Wang, E. T., Barcoded nanoparticles for high throughput in vivo discovery of targeted therapeutics. Proceedings of the National Academy of Sciences 2017, 114 (8), 2060-2065.
34. Mullard, A., DNA tags help the hunt for drugs. Nature Publishing Group: 2016.
35. Sago, C. D.; Lokugamage, M. P.; Paunovska, K.; Vanover, D. A.; Monaco, C. M.; Shah, N. N.; Castro, M. G.; Anderson, S. E.; Rudoltz, T. G.; Lando, G. N., High throughput in vivo screen of functional mRNA delivery identifies nanoparticles for endothelial cell gene editing. Proceedings of the National Academy of Sciences 2018, 115 (42), E9944-E9952.
36. Viger-Gravel, J.; Schantz, A.; Pinon, A. C.; Rossini, A. J.; Schantz, S.; Emsley, L., Structure of Lipid Nanoparticles Containing siRNA or mRNA by Dynamic Nuclear Polarization-Enhanced NMR Spectroscopy. The Journal of Physical Chemistry B 2018, 122 (7), 2073-2081.
37. Halder, K.; Benzler, M.; Hartig, J. S., Reporter assays for studying quadruplex nucleic acids. Methods 2012, 57 (1), 115-121.
38. McKinlay, C. J.; Benner, N. L.; Haabeth, O. A.; Waymouth, R. M.; Wender, P. A., Enhanced mRNA delivery into lymphocytes enabled by lipid-varied libraries of charge altering releasable transporters. Proceedings of the National Academy of Sciences 2018, 115 (26), E5859-E5866.
39. Li, B.; Dong, Y., Preparation and optimization of lipid-like nanoparticles for mRNA delivery. In RNA Nanostructures, Springer: 2017; pp 207-217.
40. Li, B.; Luo, X.; Deng, B.; Giancola, J. B.; McComb, D. W.; Schmittgen, T. D.; Dong, Y., Effects of local structural transformation of lipid-like compounds on delivery of messenger RNA. Scientific reports 2016, 6, 22137.
Miller, J. B.; Zhang, S.; Kos, P.; Xiong, H.; Zhou, K.; Perelman, S. S.; Zhu, H.; Siegwart, D. J., Non-viral CRISPR/Cas gene editing in vitro and in vivo enabled by synthetic nanoparticle co-delivery of Cas9 mRNA and sgRNA. Angewandte Chemie International Edition 2017, 56 (4), 1059-1063.
42. Karikó, K.; Muramatsu, H.; Welsh, F. A.; Ludwig, J.; Kato, H.; Akira, S.;

Weissman, D., Incorporation of pseudouridine into mRNA yields superior nonimmunogenic vector with increased translational capacity and biological stability. Molecular therapy 2008, 16 (11), 1833-1840.
43. Karikó, K.; Buckstein, M.; Ni, H.; Weissman, D., Suppression of RNA recognition by Toll-like receptors: the impact of nucleoside modification and the evolutionary origin of RNA. Immunity 2005, 23 (2), 165-175.
44. Warren, L.; Manos, P. D.; Ahfeldt, T.; Loh, Y.-H.; Li, H.; Lau, F.; Ebina, W.; Mandal, P. K.; Smith, Z. D.; Meissner, A., Highly efficient reprogramming to pluripotency and directed differentiation of human cells with synthetic modified mRNA. Cell stem cell 2010, 7 (5), 618-630.
45. Kormann, M. S.; Hasenpusch, G.; Aneja, M. K.; Nica, G.; Flemmer, A. W.; Herber-Jonat, S.; Huppmann, M.; Mays, L. E.; Illenyi, M.; Schams, A., Expression of therapeutic proteins after delivery of chemically modified mRNA in mice. Nature biotechnology 2011, 29 (2), 154.
46. Kauffman, K. J.; Dorkin, J. R.; Yang, J. H.; Heartlein, M. W.; DeRosa, F.; Mir, F. F.; Fenton, O. S.; Anderson, D. G., Optimization of lipid nanoparticle formulations for mRNA delivery in vivo with fractional factorial and definitive screening designs. Nano letters 2015, 15 (11), 7300-7306.
47. Chen, D.; Love, K. T.; Chen, Y.; Eltoukhy, A. A.; Kastrup, C.; Sahay, G.; Jeon, A.; Dong, Y.; Whitehead, K. A.; Anderson, D. G., Rapid discovery of potent siRNA containing lipid nanoparticles enabled by controlled microfluidic formulation. Journal of the American Chemical Society 2012, 134 (16), 6948-6951.
48. Ball, R. L.; Hajj, K. A.; Vizelman, J.; Bajaj, P.; Whitehead, K. A., Lipid nanoparticle formulations for enhanced co-delivery of siRNA and mRNA. Nano letters 2018.
49. Kulkarni, J. A.; Myhre, J. L.; Chen, S.; Tam, Y. Y. C.; Danescu, A.; Richman, J. M.; Cullis, P. R., Design of lipid nanoparticles for in vitro and in vivo delivery of plasmid DNA. Nanomedicine: Nanotechnology, Biology and Medicine 2017, 13 (4), 1377-1387.
50. Kulkarni, J. A.; Darjuan, M. M.; Mercer, J. E.; Chen, S.; van der Meel, R.; Thewalt, J. L.; Tam, Y. Y. C.; Cullis, P. R., On the Formation and Morphology of Lipid Nanoparticles Containing Ionizable Cationic Lipids and siRNA. ACS nano 2018, 12 (5), 4787-4795.
51. Dahlman, J. E.; Kauffman, K. J.; Xing, Y.; Shaw, T. E.; Mir, F. F.; Dlott, C. C.; Langer, R.; Anderson, D. G.; Wang, E. T., Barcoded nanoparticles for high throughput in vivo discovery of targeted therapeutics. Proceedings of the National Academy of Sciences 2017, 201620874.
52. Kenworthy, A. K.; Hristova, K.; Needham, D.; McIntosh, T. J., Range and magnitude of the steric pressure between bilayers containing phospholipids with covalently attached poly (ethylene glycol). Biophysical journal 1995, 68 (5), 1921-1936.
53. Sago, C. D.; Lokugamage, M. P.; Islam, F. Z.; Krupczak, B. R.; Sato, M.; Dahlman, J. E., Nanoparticles that deliver RNA to bone marrow identified by in vivo directed evolution. Journal of the American Chemical Society 2018, 140 (49), 17095-17105.
54. Paunovska, K.; Da Silva Sanchez, A. J.; Sago, C. D.; Gan, Z.; Lokugamage, M. P.; Islam, F. Z.; Kalathoor, S.; Krupczak, B. R.; Dahlman, J. E., Nanoparticles Containing Oxidized Cholesterol Deliver mRNA to the Liver Microenvironment at Clinically Relevant Doses. Advanced Materials 2019, 1807748.
55. Laird, P. W.; Zijderveld, A.; Linders, K.; Rudnicki, M. A.; Jaenisch, R.; Berns, A., Simplified mammalian DNA isolation procedure. Nucleic acids research 1991, 19 (15), 4293.
56. Love, K. T.; Mahon, K. P.; Levins, C. G.; Whitehead, K. A.; Querbes, W.; Dorkin, J. R.; Qin, J.; Cantley, W.; Qin, L. L.; Racie, T., Lipid-like materials for low-dose, in vivo gene silencing. Proceedings of the National Academy of Sciences 2010, 107 (5), 1864-1869.

1. A composition comprising a lipid nanoparticle (LNP) formulation comprising a LNP having encapsulated therein a barcoded mRNA (b-mRNA), wherein the b-mRNA comprises
i) a PCR handle at the 3′ UTR,
ii) a barcode sequence, and/or
iii) a unique molecular identifier (UMI).

2-3. (canceled)

4. The composition of claim 1, wherein the b-mRNA further comprises a sequence which encodes a reporter molecule.

5. (canceled)

6. The composition of claim 1 wherein the b-mRNA further comprises a sequence which encodes a biologically active molecule.

7. (canceled)

8. The composition of claim 1 wherein the b-mRNA is pseudouridine (ψ) or 5-methylcytosine (m5C) modified b-mRNA.

9-14. (canceled)

15. A composition comprising multiple LNP formulations of claim 1, wherein each LNP formulation comprises a b-mRNA having a uniquely identifiable nucleotide barcode sequence.

16. The composition of claim 15, wherein each LNP formulation is generated using a different LNP-forming composition.

17. The composition of claim 15, wherein the multiple different LNP formulations comprise b-mRNAs which differ in the coding sequence for the biologically active molecule.

18. The composition of claim 16, wherein the LNP-forming compositions vary in the molar amount and/or structure of the ionizable lipid, the molar amount and/or structure of the helper lipid, the molar amount/or structure of PEG, and/or the molar amount of cholesterol.

19. A method of analyzing in vivo delivery of a composition, the method comprising:
(i) providing at least one LNP formulation of claim to a subject; and
(ii) identifying the barcode sequence of the b-mRNA in one or more tissues of the subject,
thereby correlating the specific LNP formulation found in the tissue by the identification of the barcode.

20. The method of claim 19, further comprising:
(iii) detecting the presence of the reporter molecule in one or more tissues of the subject; and/or
(iv) quantifying the b-mRNA in the one or more tissues.

21. The method of claim 20, further comprising:
(v) sorting cells from the multiple tissues of the subject based on the presence or absence of the reporter molecule, wherein the cells having the reporter molecule are also sorted based on the presence or absence of a cell surface protein that is indicative of tissue type or cell type.

22. The method of claim 19, wherein the method comprises providing multiple different LNP formulations to the subject.

23-26. (canceled)

27. The method of claim 19, wherein the subject is a non-human mammal.

28. (canceled)

29. The method of claim 19, wherein greater than 100, 500, 1000, 5000 or 10000 different LNP formulations are assayed.

30. The method of claim 29, which is done in high-throughput format.

31. The method of claim 19, wherein the quantifying is done using deep sequencing.

32. The method of claim 19, wherein the LNP are provided to the subject orally, intramuscularly, intravenously, or intracranially.

33. A method of determining a personalized treatment for a subject, the method comprising
(i) obtaining a biological sample from the subject, said sample containing one or more cells;
(ii) providing at least one LNP formulation of claim to the sample; and
(iii) identifying the barcode sequence of the b-mRNA in the one or more cells,
thereby correlating the specific LNP formulation found in the tissue by the identification of the barcode.

34. The method of 33, wherein the sample is a tissue sample, a tumor biopsy, or a blood sample.

35-36. (canceled)


SEQUENCE LISTING (151 sequences)

SEQ ID NO: 1 - DNA, 37 bp, Artificial Sequence
  synthetic construct
taatacgactcactatagggcattccggtactgttgg

SEQ ID NO: 2 - DNA, 50 bp, Artificial Sequence
  synthetic construct
  misc_feature (21)..(30): n is a, c, g, or t
gcccagtcatagccgaatagnnnnnnnnnnccgccccgactctagaatta

SEQ ID NO: 3 - DNA, 54 bp, Artificial Sequence
  synthetic construct
caagcagaagacggcatacgagatcctggtaggtgactggagttcagacgtgtg

SEQ ID NO: 4 - DNA, 54 bp, Artificial Sequence
  synthetic construct
caagcagaagacggcatacgagattaagcatggtgactggagttcagacgtgtg

SEQ ID NO: 5 - DNA, 54 bp, Artificial Sequence
  synthetic construct
caagcagaagacggcatacgagatagatgtgcgtgactggagttcagacgtgtg

SEQ ID NO: 6 - DNA, 54 bp, Artificial Sequence
  synthetic construct
caagcagaagacggcatacgagatgtcgagcagtgactggagttcagacgtgtg

SEQ ID NO: 7 - DNA, 54 bp, Artificial Sequence
  synthetic construct
caagcagaagacggcatacgagatgaattgctgtgactggagttcagacgtgtg

SEQ ID NO: 8 - DNA, 54 bp, Artificial Sequence
  synthetic construct
caagcagaagacggcatacgagataagcaactgtgactggagttcagacgtgtg

SEQ ID NO: 9 - DNA, 54 bp, Artificial Sequence
  synthetic construct
caagcagaagacggcatacgagatctaactgggtgactggagttcagacgtgtg

SEQ ID NO: 10 - DNA, 54 bp, Artificial Sequence
  synthetic construct
caagcagaagacggcatacgagataggctcaagtgactggagttcagacgtgtg

SEQ ID NO: 11 - DNA, 54 bp, Artificial Sequence
  synthetic construct
caagcagaagacggcatacgagatcagttggtgtgactggagttcagacgtgtg

SEQ ID NO: 12 - DNA, 54 bp, Artificial Sequence
  synthetic construct
caagcagaagacggcatacgagattctggaccgtgactggagttcagacgtgtg

SEQ ID NO: 13 - DNA, 54 bp, Artificial Sequence
  synthetic construct
caagcagaagacggcatacgagattgttatacgtgactggagttcagacgtgtg

SEQ ID NO: 14 - DNA, 54 bp, Artificial Sequence
  synthetic construct
caagcagaagacggcatacgagattcagcgaagtgactggagttcagacgtgtg

SEQ ID NO: 15 - DNA, 54 bp, Artificial Sequence
  synthetic construct
caagcagaagacggcatacgagatgtcaagttgtgactggagttcagacgtgtg

SEQ ID NO: 16 - DNA, 54 bp, Artificial Sequence
  synthetic construct
caagcagaagacggcatacgagataggatgtggtgactggagttcagacgtgtg

SEQ ID NO: 17 - DNA, 54 bp, Artificial Sequence
  synthetic construct
caagcagaagacggcatacgagatcattccgagtgactggagttcagacgtgtg

SEQ ID NO: 18 - DNA, 54 bp, Artificial Sequence
  synthetic construct
caagcagaagacggcatacgagatacatccttgtgactggagttcagacgtgtg

SEQ ID NO: 19 - DNA, 54 bp, Artificial Sequence
  synthetic construct
caagcagaagacggcatacgagattcgtgtgcgtgactggagttcagacgtgtg

SEQ ID NO: 20 - DNA, 54 bp, Artificial Sequence
  synthetic construct
caagcagaagacggcatacgagattcgccagagtgactggagttcagacgtgtg

SEQ ID NO: 21 - DNA, 54 bp, Artificial Sequence
  synthetic construct
caagcagaagacggcatacgagattcgctatggtgactggagttcagacgtgtg

SEQ ID NO: 22 - DNA, 54 bp, Artificial Sequence
  synthetic construct
caagcagaagacggcatacgagatggctcctggtgactggagttcagacgtgtg

SEQ ID NO: 23 - DNA, 54 bp, Artificial Sequence
  synthetic construct
caagcagaagacggcatacgagatatccgacagtgactggagttcagacgtgtg

SEQ ID NO: 24 - DNA, 54 bp, Artificial Sequence
  synthetic construct
caagcagaagacggcatacgagataacataatgtgactggagttcagacgtgtg

SEQ ID NO: 25 - DNA, 54 bp, Artificial Sequence
  synthetic construct
caagcagaagacggcatacgagatatggtagggtgactggagttcagacgtgtg

SEQ ID NO: 26 - DNA, 54 bp, Artificial Sequence
  synthetic construct
caagcagaagacggcatacgagatgctaagtagtgactggagttcagacgtgtg

SEQ ID NO: 27 - DNA, 54 bp, Artificial Sequence
  synthetic construct
caagcagaagacggcatacgagatacttcttcgtgactggagttcagacgtgtg

SEQ ID NO: 28 - DNA, 54 bp, Artificial Sequence
  synthetic construct
caagcagaagacggcatacgagattagatcctgtgactggagttcagacgtgtg

SEQ ID NO: 29 - DNA, 54 bp, Artificial Sequence
  synthetic construct
caagcagaagacggcatacgagatttactgtcgtgactggagttcagacgtgtg

SEQ ID NO: 30 - DNA, 54 bp, Artificial Sequence
  synthetic construct
caagcagaagacggcatacgagatggcatagggtgactggagttcagacgtgtg

SEQ ID NO: 31 - DNA, 54 bp, Artificial Sequence
  synthetic construct
caagcagaagacggcatacgagatcaaggcgagtgactggagttcagacgtgtg

SEQ ID NO: 32 - DNA, 54 bp, Artificial Sequence
  synthetic construct
caagcagaagacggcatacgagatgacgctatgtgactggagttcagacgtgtg

SEQ ID NO: 33 - DNA, 54 bp, Artificial Sequence
  synthetic construct
caagcagaagacggcatacgagataaggcgacgtgactggagttcagacgtgtg

SEQ ID NO: 34 - DNA, 54 bp, Artificial Sequence
  synthetic construct
caagcagaagacggcatacgagatcctagaatgtgactggagttcagacgtgtg

SEQ ID NO: 35 - DNA, 54 bp, Artificial Sequence
  synthetic construct
caagcagaagacggcatacgagattggtaacggtgactggagttcagacgtgtg

SEQ ID NO: 36 - DNA, 54 bp, Artificial Sequence
  synthetic construct
caagcagaagacggcatacgagatcatcagacgtgactggagttcagacgtgtg

SEQ ID NO: 37 - DNA, 54 bp, Artificial Sequence
  synthetic construct
caagcagaagacggcatacgagatgtgcgtaagtgactggagttcagacgtgtg

SEQ ID NO: 38 - DNA, 54 bp, Artificial Sequence
  synthetic construct
caagcagaagacggcatacgagatctattcaagtgactggagttcagacgtgtg

SEQ ID NO: 39 - DNA, 54 bp, Artificial Sequence
  synthetic construct
caagcagaagacggcatacgagatagtgtcttgtgactggagttcagacgtgtg

SEQ ID NO: 40 - DNA, 54 bp, Artificial Sequence
  synthetic construct
caagcagaagacggcatacgagatccttgctggtgactggagttcagacgtgtg

SEQ ID NO: 41 - DNA, 54 bp, Artificial Sequence
  synthetic construct
caagcagaagacggcatacgagatttgctggagtgactggagttcagacgtgtg

SEQ ID NO: 42 - DNA, 54 bp, Artificial Sequence
  synthetic construct
caagcagaagacggcatacgagatagctctgggtgactggagttcagacgtgtg

SEQ ID NO: 43 - DNA, 54 bp, Artificial Sequence
  synthetic construct
caagcagaagacggcatacgagataccaaggagtgactggagttcagacgtgtg

SEQ ID NO: 44 - DNA, 54 bp, Artificial Sequence
  synthetic construct
caagcagaagacggcatacgagatgataacctgtgactggagttcagacgtgtg

SEQ ID NO: 45 - DNA, 54 bp, Artificial Sequence
  synthetic construct
caagcagaagacggcatacgagattagatgacgtgactggagttcagacgtgtg

SEQ ID NO: 46 - DNA, 54 bp, Artificial Sequence
  synthetic construct
caagcagaagacggcatacgagattgcgaagggtgactggagttcagacgtgtg

SEQ ID NO: 47 - DNA, 54 bp, Artificial Sequence
  synthetic construct
caagcagaagacggcatacgagatgaccgagagtgactggagttcagacgtgtg

SEQ ID NO: 48 - DNA, 54 bp, Artificial Sequence
  synthetic construct
caagcagaagacggcatacgagatcagacaatgtgactggagttcagacgtgtg

SEQ ID NO: 49 - DNA, 54 bp, Artificial Sequence
  synthetic construct
caagcagaagacggcatacgagatctaggttcgtgactggagttcagacgtgtg

SEQ ID NO: 50 - DNA, 54 bp, Artificial Sequence
  synthetic construct
caagcagaagacggcatacgagatgttcattagtgactggagttcagacgtgtg

SEQ ID NO: 51 - DNA, 60 bp, Artificial Sequence
  synthetic construct
  misc_feature (21)..(30): n is a, c, g, or t
gcccagtcatagccgaatagnnnnnnnnnnaagtaccctcccgccccgactctagaatta

SEQ ID NO: 52 - DNA, 60 bp, Artificial Sequence
  synthetic construct
  misc_feature (21)..(30): n is a, c, g, or t
gcccagtcatagccgaatagnnnnnnnnnnggcaattgtcccgccccgactctagaatta

SEQ ID NO: 53 - DNA, 60 bp, Artificial Sequence
  synthetic construct
  misc_feature (21)..(30): n is a, c, g, or t
gcccagtcatagccgaatagnnnnnnnnnnaggtgcgttaccgccccgactctagaatta

SEQ ID NO: 54 - DNA, 60 bp, Artificial Sequence
  synthetic construct
  misc_feature (21)..(30): n is a, c, g, or t
gcccagtcatagccgaatagnnnnnnnnnncgacgatcatccgccccgactctagaatta

SEQ ID NO: 55 - DNA, 60 bp, Artificial Sequence
  synthetic construct
  misc_feature (21)..(30): n is a, c, g, or t
gcccagtcatagccgaatagnnnnnnnnnnatgggagacaccgccccgactctagaatta

SEQ ID NO: 56 - DNA, 60 bp, Artificial Sequence
  synthetic construct
  misc_feature (21)..(30): n is a, c, g, or t
gcccagtcatagccgaatagnnnnnnnnnnctgtttctccccgccccgactctagaatta

SEQ ID NO: 57 - DNA, 60 bp, Artificial Sequence
  synthetic construct
  misc_feature (21)..(30): n is a, c, g, or t
gcccagtcatagccgaatagnnnnnnnnnncgtttgtacgccgccccgactctagaatta

SEQ ID NO: 58 - DNA, 60 bp, Artificial Sequence
  synthetic construct
  misc_feature (21)..(30): n is a, c, g, or t
gcccagtcatagccgaatagnnnnnnnnnncccacaaatcccgccccgactctagaatta

SEQ ID NO: 59 - DNA, 60 bp, Artificial Sequence
  synthetic construct
  misc_feature (21)..(30): n is a, c, g, or t
gcccagtcatagccgaatagnnnnnnnnnnaaggctgcaaccgccccgactctagaatta

SEQ ID NO: 60 - DNA, 60 bp, Artificial Sequence
  synthetic construct
  misc_feature (21)..(30): n is a, c, g, or t
gcccagtcatagccgaatagnnnnnnnnnngtcagcattcccgccccgactctagaatta

SEQ ID NO: 61 - DNA, 60 bp, Artificial Sequence
  synthetic construct
  misc_feature (21)..(30): n is a, c, g, or t
gcccagtcatagccgaatagnnnnnnnnnncctcatggatccgccccgactctagaatta

SEQ ID NO: 62 - DNA, 60 bp, Artificial Sequence
  synthetic construct
  misc_feature (21)..(30): n is a, c, g, or t
gcccagtcatagccgaatagnnnnnnnnnncatcgtggaaccgccccgactctagaatta

SEQ ID NO: 63 - DNA, 60 bp, Artificial Sequence
  synthetic construct
  misc_feature (21)..(30): n is a, c, g, or t
gcccagtcatagccgaatagnnnnnnnnnnaattcccagcccgccccgactctagaatta

SEQ ID NO: 64 - DNA, 60 bp, Artificial Sequence
  synthetic construct
  misc_feature (21)..(30): n is a, c, g, or t
gcccagtcatagccgaatagnnnnnnnnnncgtcgttttgccgccccgactctagaatta

SEQ ID NO: 65 - DNA, 60 bp, Artificial Sequence
  synthetic construct
  misc_feature (21)..(30): n is a, c, g, or t
gcccagtcatagccgaatagnnnnnnnnnnaaaggcgagaccgccccgactctagaatta

SEQ ID NO: 66 - DNA, 60 bp, Artificial Sequence
  synthetic construct
  misc_feature (21)..(30): n is a, c, g, or t
gcccagtcatagccgaatagnnnnnnnnnnctctgatctgccgccccgactctagaatta

SEQ ID NO: 67 - DNA, 60 bp, Artificial Sequence
  synthetic construct
  misc_feature (21)..(30): n is a, c, g, or t
gcccagtcatagccgaatagnnnnnnnnnnagaacgtgtcccgccccgactctagaatta

SEQ ID NO: 68 - DNA, 60 bp, Artificial Sequence
  synthetic construct
  misc_feature (21)..(30): n is a, c, g, or t
gcccagtcatagccgaatagnnnnnnnnnnagaccttagcccgccccgactctagaatta

SEQ ID NO: 69 - DNA, 60 bp, Artificial Sequence
  synthetic construct
  misc_feature (21)..(30): n is a, c, g, or t
gcccagtcatagccgaatagnnnnnnnnnnaggtcgaacaccgccccgactctagaatta

SEQ ID NO: 70 - DNA, 60 bp, Artificial Sequence
  synthetic construct
  misc_feature (21)..(30): n is a, c, g, or t
gcccagtcatagccgaatagnnnnnnnnnntgtctttgggccgccccgactctagaatta

SEQ ID NO: 71 - DNA, 60 bp, Artificial Sequence
  synthetic construct
  misc_feature (21)..(30): n is a, c, g, or t
gcccagtcatagccgaatagnnnnnnnnnnttcctacctgccgccccgactctagaatta

SEQ ID NO: 72 - DNA, 60 bp, Artificial Sequence
  synthetic construct
  misc_feature (21)..(30): n is a, c, g, or t
gcccagtcatagccgaatagnnnnnnnnnncgcgataatgccgccccgactctagaatta

SEQ ID NO: 73 - DNA, 60 bp, Artificial Sequence
  synthetic construct
  misc_feature (21)..(30): n is a, c, g, or t
gcccagtcatagccgaatagnnnnnnnnnntcagtctctgccgccccgactctagaatta

SEQ ID NO: 74 - DNA, 60 bp, Artificial Sequence
  synthetic construct
  misc_feature (21)..(30): n is a, c, g, or t
gcccagtcatagccgaatagnnnnnnnnnngtgcatgtcaccgccccgactctagaatta

SEQ ID NO: 75 - DNA, 60 bp, Artificial Sequence
  synthetic construct
  misc_feature (21)..(30): n is a, c, g, or t
gcccagtcatagccgaatagnnnnnnnnnnggaaccaacaccgccccgactctagaatta

SEQ ID NO: 76 - DNA, 60 bp, Artificial Sequence
  synthetic construct
  misc_feature (21)..(30): n is a, c, g, or t
gcccagtcatagccgaatagnnnnnnnnnngttcagagagccgccccgactctagaatta

SEQ ID NO: 77 - DNA, 60 bp, Artificial Sequence
  synthetic construct
  misc_feature (21)..(30): n is a, c, g, or t
gcccagtcatagccgaatagnnnnnnnnnnttggctagtgccgccccgactctagaatta

SEQ ID NO: 78 - DNA, 60 bp, Artificial Sequence
  synthetic construct
  misc_feature (21)..(30): n is a, c, g, or t
gcccagtcatagccgaatagnnnnnnnnnnggcaaagtcaccgccccgactctagaatta

SEQ ID NO: 79 - DNA, 60 bp, Artificial Sequence
  synthetic construct
  misc_feature (21)..(30): n is a, c, g, or t
gcccagtcatagccgaatagnnnnnnnnnncttcgctgaaccgccccgactctagaatta

SEQ ID NO: 80 - DNA, 60 bp, Artificial Sequence
  synthetic construct
  misc_feature (21)..(30): n is a, c, g, or t
gcccagtcatagccgaatagnnnnnnnnnngtatgcctgtccgccccgactctagaatta

SEQ ID NO: 81 - DNA, 60 bp, Artificial Sequence
  synthetic construct
  misc_feature (21)..(30): n is a, c, g, or t
gcccagtcatagccgaatagnnnnnnnnnnttaggagtcgccgccccgactctagaatta

SEQ ID NO: 82 - DNA, 60 bp, Artificial Sequence
  synthetic construct
  misc_feature (21)..(30): n is a, c, g, or t
gcccagtcatagccgaatagnnnnnnnnnnagatagcgtgccgccccgactctagaatta

SEQ ID NO: 83 - DNA, 60 bp, Artificial Sequence
  synthetic construct
  misc_feature (21)..(30): n is a, c, g, or t
gcccagtcatagccgaatagnnnnnnnnnnaagcggacttccgccccgactctagaatta

SEQ ID NO: 84 - DNA, 60 bp, Artificial Sequence
  synthetic construct
  misc_feature (21)..(30): n is a, c, g, or t
gcccagtcatagccgaatagnnnnnnnnnncggtctagatccgccccgactctagaatta

SEQ ID NO: 85 - DNA, 60 bp, Artificial Sequence
  synthetic construct
  misc_feature (21)..(30): n is a, c, g, or t
gcccagtcatagccgaatagnnnnnnnnnnaagggggattccgccccgactctagaatta

SEQ ID NO: 86 - DNA, 60 bp, Artificial Sequence
  synthetic construct
  misc_feature (21)..(30): n is a, c, g, or t
gcccagtcatagccgaatagnnnnnnnnnncagatgcgaaccgccccgactctagaatta

SEQ ID NO: 87 - DNA, 60 bp, Artificial Sequence
  synthetic construct
  misc_feature (21)..(30): n is a, c, g, or t
gcccagtcatagccgaatagnnnnnnnnnnggaactccaaccgccccgactctagaatta

SEQ ID NO: 88 - DNA, 60 bp, Artificial Sequence
  synthetic construct
  misc_feature (21)..(30): n is a, c, g, or t
gcccagtcatagccgaatagnnnnnnnnnngattggccaaccgccccgactctagaatta

SEQ ID NO: 89 - DNA, 60 bp, Artificial Sequence
  synthetic construct
  misc_feature (21)..(30): n is a, c, g, or t
gcccagtcatagccgaatagnnnnnnnnnntgtcgcctttccgccccgactctagaatta

SEQ ID NO: 90 - DNA, 60 bp, Artificial Sequence
  synthetic construct
  misc_feature (21)..(30): n is a, c, g, or t
gcccagtcatagccgaatagnnnnnnnnnncagcgacattccgccccgactctagaatta

SEQ ID NO: 91 - DNA, 60 bp, Artificial Sequence
  synthetic construct
  misc_feature (21)..(30): n is a, c, g, or t
gcccagtcatagccgaatagnnnnnnnnnnccatacgttgccgccccgactctagaatta

SEQ ID NO: 92 - DNA, 60 bp, Artificial Sequence
  synthetic construct
  misc_feature (21)..(30): n is a, c, g, or t
gcccagtcatagccgaatagnnnnnnnnnnggttaggtagccgccccgactctagaatta

SEQ ID NO: 93 - DNA, 60 bp, Artificial Sequence
  synthetic construct
  misc_feature (21)..(30): n is a, c, g, or t
gcccagtcatagccgaatagnnnnnnnnnntacaccgtgaccgccccgactctagaatta

SEQ ID NO: 94 - DNA, 60 bp, Artificial Sequence
  synthetic construct
  misc_feature (21)..(30): n is a, c, g, or t
gcccagtcatagccgaatagnnnnnnnnnntacttctgggccgccccgactctagaatta

SEQ ID NO: 95 - DNA, 60 bp, Artificial Sequence
  synthetic construct
  misc_feature (21)..(30): n is a, c, g, or t
gcccagtcatagccgaatagnnnnnnnnnnaagcgcttgtccgccccgactctagaatta

SEQ ID NO: 96 - DNA, 60 bp, Artificial Sequence
  synthetic construct
  misc_feature (21)..(30): n is a, c, g, or t
gcccagtcatagccgaatagnnnnnnnnnncggttaatccccgccccgactctagaatta

SEQ ID NO: 97 - DNA, 60 bp, Artificial Sequence
  synthetic construct
  misc_feature (21)..(30): n is a, c, g, or t
gcccagtcatagccgaatagnnnnnnnnnntatcacgaggccgccccgactctagaatta

SEQ ID NO: 98 - DNA, 60 bp, Artificial Sequence
  synthetic construct
  misc_feature (21)..(30): n is a, c, g, or t
gcccagtcatagccgaatagnnnnnnnnnnccgaatagtcccgccccgactctagaatta

SEQ ID NO: 99 - DNA, 37 bp, Artificial Sequence; identical to SEQ ID NO: 1
  synthetic construct

SEQ ID NO: 100 - DNA, 41 bp, Artificial Sequence
  synthetic construct
agacgtgtgctcttccgatctggacgaggtgcctaaaggac

SEQ ID NO: 101 - DNA, 40 bp, Artificial Sequence
  synthetic construct
acacgacgctcttccgatctgcccagtcatagccgaatag

SEQ ID NO: 102 - DNA, 58 bp, Artificial Sequence
  synthetic construct
aatgatacggcgaccaccgagatctacactctttccctacacgacgctcttccgatct

SEQ ID NO: 103 - DNA, 33 bp, Artificial Sequence
  synthetic construct
tgactggagttcagacgtgtgctcttccgatct

SEQ ID NO: 104 - DNA, 61 bp, Artificial Sequence
  synthetic construct
  misc_feature (32)..(41): n is a, c, g, or t
agacgtgtgctcttccgatctgagggtacttnnnnnnnnnnagatcggaagagcgtcgtgt

SEQ ID NO: 105 - DNA, 61 bp, Artificial Sequence
  synthetic construct
  misc_feature (32)..(41): n is a, c, g, or t
agacgtgtgctcttccgatctgacaattgccnnnnnnnnnnagatcggaagagcgtcgtgt

SEQ ID NO: 106 - DNA, 61 bp, Artificial Sequence
  synthetic construct
  misc_feature (32)..(41): n is a, c, g, or t
agacgtgtgctcttccgatcttaacgcacctnnnnnnnnnnagatcggaagagcgtcgtgt

SEQ ID NO: 107 - DNA, 61 bp, Artificial Sequence
  synthetic construct
  misc_feature (32)..(41): n is a, c, g, or t
agacgtgtgctcttccgatctatgatcgtcgnnnnnnnnnnagatcggaagagcgtcgtgt

SEQ ID NO: 108 - DNA, 61 bp, Artificial Sequence
  synthetic construct
  misc_feature (32)..(41): n is a, c, g, or t
agacgtgtgctcttccgatcttgtctcccatnnnnnnnnnnagatcggaagagcgtcgtgt

SEQ ID NO: 109 - DNA, 61 bp, Artificial Sequence
  synthetic construct
  misc_feature (32)..(41): n is a, c, g, or t
agacgtgtgctcttccgatctggagaaacagnnnnnnnnnnagatcggaagagcgtcgtgt

SEQ ID NO: 110 - DNA, 61 bp, Artificial Sequence
  synthetic construct
  misc_feature (32)..(41): n is a, c, g, or t
agacgtgtgctcttccgatctcgtacaaacgnnnnnnnnnnagatcggaagagcgtcgtgt

SEQ ID NO: 111 - DNA, 61 bp, Artificial Sequence
  synthetic construct
  misc_feature (32)..(41): n is a, c, g, or t
agacgtgtgctcttccgatctgatttgtgggnnnnnnnnnnagatcggaagagcgtcgtgt

SEQ ID NO: 112 - DNA, 61 bp, Artificial Sequence
  synthetic construct
  misc_feature (32)..(41): n is a, c, g, or t
agacgtgtgctcttccgatctttgcagccttnnnnnnnnnnagatcggaagagcgtcgtgt

SEQ ID NO: 113 - DNA, 61 bp, Artificial Sequence
  synthetic construct
  misc_feature (32)..(41): n is a, c, g, or t
agacgtgtgctcttccgatctgaatgctgacnnnnnnnnnnagatcggaagagcgtcgtgt

SEQ ID NO: 114 - DNA, 61 bp, Artificial Sequence
  synthetic construct
  misc_feature (32)..(41): n is a, c, g, or t
agacgtgtgctcttccgatctatccatgaggnnnnnnnnnnagatcggaagagcgtcgtgt

SEQ ID NO: 115 - DNA, 61 bp, Artificial Sequence
  synthetic construct
  misc_feature (32)..(41): n is a, c, g, or t
agacgtgtgctcttccgatctttccacgatgnnnnnnnnnnagatcggaagagcgtcgtgt

SEQ ID NO: 116 - DNA, 61 bp, Artificial Sequence
  synthetic construct
  misc_feature (32)..(41): n is a, c, g, or t
agacgtgtgctcttccgatctgctgggaattnnnnnnnnnnagatcggaagagcgtcgtgt

SEQ ID NO: 117 - DNA, 61 bp, Artificial Sequence
  synthetic construct
  misc_feature (32)..(41): n is a, c, g, or t
agacgtgtgctcttccgatctcaaaacgacgnnnnnnnnnnagatcggaagagcgtcgtgt

SEQ ID NO: 118 - DNA, 61 bp, Artificial Sequence
  synthetic construct
  misc_feature (32)..(41): n is a, c, g, or t
agacgtgtgctcttccgatcttctcgcctttnnnnnnnnnnagatcggaagagcgtcgtgt

SEQ ID NO: 119 - DNA, 61 bp, Artificial Sequence
  synthetic construct
  misc_feature (32)..(41): n is a, c, g, or t
agacgtgtgctcttccgatctcagatcagagnnnnnnnnnnagatcggaagagcgtcgtgt

SEQ ID NO: 120 - DNA, 61 bp, Artificial Sequence
  synthetic construct
  misc_feature (32)..(41): n is a, c, g, or t
agacgtgtgctcttccgatctgacacgttctnnnnnnnnnnagatcggaagagcgtcgtgt

SEQ ID NO: 121 - DNA, 61 bp, Artificial Sequence
  synthetic construct
  misc_feature (32)..(41): n is a, c, g, or t
agacgtgtgctcttccgatctgctaaggtctnnnnnnnnnnagatcggaagagcgtcgtgt

SEQ ID NO: 122 - DNA, 61 bp, Artificial Sequence
  synthetic construct
  misc_feature (32)..(41): n is a, c, g, or t
agacgtgtgctcttccgatcttgttcgacctnnnnnnnnnnagatcggaagagcgtcgtgt

SEQ ID NO: 123 - DNA, 61 bp, Artificial Sequence
  synthetic construct
  misc_feature (32)..(41): n is a, c, g, or t
agacgtgtgctcttccgatctcccaaagacannnnnnnnnnagatcggaagagcgtcgtgt

SEQ ID NO: 124 - DNA, 61 bp, Artificial Sequence
  synthetic construct
  misc_feature (32)..(41): n is a, c, g, or t
agacgtgtgctcttccgatctcaggtaggaannnnnnnnnnagatcggaagagcgtcgtgt

SEQ ID NO: 125 - DNA, 61 bp, Artificial Sequence
  synthetic construct
  misc_feature (32)..(41): n is a, c, g, or t
agacgtgtgctcttccgatctcattatcgcgnnnnnnnnnnagatcggaagagcgtcgtgt

SEQ ID NO: 126 - DNA, 61 bp, Artificial Sequence
  synthetic construct
  misc_feature (32)..(41): n is a, c, g, or t
agacgtgtgctcttccgatctcagagactgannnnnnnnnnagatcggaagagcgtcgtgt

SEQ ID NO: 127 - DNA, 61 bp, Artificial Sequence
  synthetic construct
  misc_feature (32)..(41): n is a, c, g, or t
agacgtgtgctcttccgatcttgacatgcacnnnnnnnnnnagatcggaagagcgtcgtgt

SEQ ID NO: 128 - DNA, 61 bp, Artificial Sequence
  synthetic construct
  misc_feature (32)..(41): n is a, c, g, or t
agacgtgtgctcttccgatcttgttggttccnnnnnnnnnnagatcggaagagcgtcgtgt

SEQ ID NO: 129 - DNA, 61 bp, Artificial Sequence
  synthetic construct
  misc_feature (32)..(41): n is a, c, g, or t
agacgtgtgctcttccgatctctctctgaacnnnnnnnnnnagatcggaagagcgtcgtgt

SEQ ID NO: 130 - DNA, 61 bp, Artificial Sequence
  synthetic construct
  misc_feature (32)..(41): n is a, c, g, or t
agacgtgtgctcttccgatctcactagccaannnnnnnnnnagatcggaagagcgtcgtgt

SEQ ID NO: 131 - DNA, 61 bp, Artificial Sequence
  synthetic construct
  misc_feature (32)..(41): n is a, c, g, or t
agacgtgtgctcttccgatcttgactttgccnnnnnnnnnnagatcggaagagcgtcgtgt

SEQ ID NO: 132 - DNA, 61 bp, Artificial Sequence
  synthetic construct
  misc_feature (32)..(41): n is a, c, g, or t
agacgtgtgctcttccgatctttcagcgaagnnnnnnnnnnagatcggaagagcgtcgtgt

SEQ ID NO: 133 - DNA, 61 bp, Artificial Sequence
  synthetic construct
  misc_feature (32)..(41): n is a, c, g, or t
agacgtgtgctcttccgatctacaggcatacnnnnnnnnnnagatcggaagagcgtcgtgt

SEQ ID NO: 134 - DNA, 61 bp, Artificial Sequence
  synthetic construct
  misc_feature (32)..(41): n is a, c, g, or t
agacgtgtgctcttccgatctcgactcctaannnnnnnnnnagatcggaagagcgtcgtgt

SEQ ID NO: 135 - DNA, 61 bp, Artificial Sequence
  synthetic construct
  misc_feature (32)..(41): n is a, c, g, or t
agacgtgtgctcttccgatctcacgctatctnnnnnnnnnnagatcggaagagcgtcgtgt

SEQ ID NO: 136 - DNA, 61 bp, Artificial Sequence
  synthetic construct
  misc_feature (32)..(41): n is a, c, g, or t
agacgtgtgctcttccgatctaagtccgcttnnnnnnnnnnagatcggaagagcgtcgtgt

SEQ ID NO: 137 - DNA, 61 bp, Artificial Sequence
  synthetic construct
  misc_feature (32)..(41): n is a, c, g, or t
agacgtgtgctcttccgatctatctagaccgnnnnnnnnnnagatcggaagagcgtcgtgt

SEQ ID NO: 138 - DNA, 61 bp, Artificial Sequence
  synthetic construct
  misc_feature (32)..(41): n is a, c, g, or t
agacgtgtgctcttccgatctaatcccccttnnnnnnnnnnagatcggaagagcgtcgtgt

SEQ ID NO: 139 - DNA, 61 bp, Artificial Sequence
  synthetic construct
  misc_feature (32)..(41): n is a, c, g, or t
agacgtgtgctcttccgatctttcgcatctgnnnnnnnnnnagatcggaagagcgtcgtgt

SEQ ID NO: 140 - DNA, 61 bp, Artificial Sequence
  synthetic construct
  misc_feature (32)..(41): n is a, c, g, or t
agacgtgtgctcttccgatctttggagttccnnnnnnnnnnagatcggaagagcgtcgtgt

SEQ ID NO: 141 - DNA, 61 bp, Artificial Sequence
  synthetic construct
  misc_feature (32)..(41): n is a, c, g, or t
agacgtgtgctcttccgatctttggccaatcnnnnnnnnnnagatcggaagagcgtcgtgt

SEQ ID NO: 142 - DNA, 61 bp, Artificial Sequence
  synthetic construct
  misc_feature (32)..(41): n is a, c, g, or t
agacgtgtgctcttccgatctaaaggcgacannnnnnnnnnagatcggaagagcgtcgtgt

SEQ ID NO: 143 - DNA, 61 bp, Artificial Sequence
  synthetic construct
  misc_feature (32)..(41): n is a, c, g, or t
agacgtgtgctcttccgatctaatgtcgctgnnnnnnnnnnagatcggaagagcgtcgtgt

SEQ ID NO: 144 - DNA, 61 bp, Artificial Sequence
  synthetic construct
  misc_feature (32)..(41): n is a, c, g, or t
agacgtgtgctcttccgatctcaacgtatggnnnnnnnnnnagatcggaagagcgtcgtgt

SEQ ID NO: 145 - DNA, 61 bp, Artificial Sequence
  synthetic construct
  misc_feature (32)..(41): n is a, c, g, or t
agacgtgtgctcttccgatctctacctaaccnnnnnnnnnnagatcggaagagcgtcgtgt

SEQ ID NO: 146 - DNA, 61 bp, Artificial Sequence
  synthetic construct
  misc_feature (32)..(41): n is a, c, g, or t
agacgtgtgctcttccgatcttcacggtgtannnnnnnnnnagatcggaagagcgtcgtgt

SEQ ID NO: 147 - DNA, 61 bp, Artificial Sequence
  synthetic construct
  misc_feature (32)..(41): n is a, c, g, or t
agacgtgtgctcttccgatctcccagaagtannnnnnnnnnagatcggaagagcgtcgtgt

SEQ ID NO: 148 - DNA, 61 bp, Artificial Sequence
  synthetic construct
  misc_feature (32)..(41): n is a, c, g, or t
agacgtgtgctcttccgatctacaagcgcttnnnnnnnnnnagatcggaagagcgtcgtgt

SEQ ID NO: 149 - DNA, 61 bp, Artificial Sequence
  synthetic construct
  misc_feature (32)..(41): n is a, c, g, or t
agacgtgtgctcttccgatctggattaaccgnnnnnnnnnnagatcggaagagcgtcgtgt

SEQ ID NO: 150 - DNA, 61 bp, Artificial Sequence
  synthetic construct
  misc_feature (32)..(41): n is a, c, g, or t
agacgtgtgctcttccgatctcctcgtgatannnnnnnnnnagatcggaagagcgtcgtgt

SEQ ID NO: 151 - DNA, 61 bp, Artificial Sequence
  synthetic construct
  misc_feature (32)..(41): n is a, c, g, or t
agacgtgtgctcttccgatctgactattcggnnnnnnnnnnagatcggaagagcgtcgtgt